(19) United States
(12) Patent Application Publication
(10) Pub. No.: US 2017/0245495 A1
(43) Pub. Date: Aug. 31, 2017

(54) CHEMICAL LURE FOR ASIAN CITRUS PSYLLID

(71) Applicants: The Regents of the University of California, Oakland, CA (US); University of Florida Research Foundation, Inc., Gainesville, FL (US)

(72) Inventors: Cristina E. DAVIS, Davis, CA (US); Alexander AKSENOV, Davis, CA (US); Lukasz STELINSKI, Gainesville, FL (US); Xavier MARTINI, Gainesville, FL (US)

(21) Appl. No.: 15/521,838

(22) PCT Filed: Oct. 27, 2015
(86) PCT No.: PCT/US2015/057659

§ 371 Date: Apr. 25, 2017

Related U.S. Application Data
(60) Provisional application No. 62/069,199, filed on Oct. 27, 2014.

(51) Int. Cl. A01N 27/00; A01N 35/02; G01N 30/72; A01N 37/40; A01N 49/00; C12Q 1/06; A01N 31/02; A01N 37/02

(52) U.S. Cl. CPC A01N 27/00; A01N 31/02; A01N 35/02; A01N 37/02; A01N 37/40; A01N 49/00; C12Q 1/06; G01N 30/7206; A01N 2300/00; C12Q 1/686

(57) ABSTRACT

The present disclosure relates to compositions containing a mixture of e.g. one or more or two or more compounds released by a citrus plant in quantities that are altered during infection with Huanglongbing disease, where the composition is an attractant for psyllids. Furthermore, compositions contain one or more active compounds which constitute a synthetic chemical blend for attracting psyllids. Active compounds in the compositions may include, for example, one or more compounds selected from linalool, tridecane, 4-OH-4-Me-2-pentanone, hexacosane, 1-tetradecene, tricosane, geranial, tetradecanal, phenylacetaldehyde, methyl salicylate, cumacrene, (E)-beta-ocimene, hexadecanol, and geranyl acetone.

### CROSS-REFERENCE TO RELATED APPLICATION

This application claims the benefit of U.S. Provisional Application No. 62/069,199 filed on Oct. 27, 2014, which is incorporated herein by reference in its entirety.

### FIELD

The present disclosure relates to chemical lures for Diaphorina citri (Asian citrus psyllid) and methods of using these chemical lures.

### BACKGROUND

Plants are known to communicate with one another and with insects by emitting bouquets of volatile chemicals called volatile organic compounds (VOCs). These chemical cues are released, in some cases, in response to damage by insects (Kost and Heil, 2006). Plant-insect ‘conversations’ have been investigated for approximately two decades and a great deal of progress has been made. It is now clear that different plant species emit their own unique chemical blends and some chemicals have ubiquitous importance. Examples include methyl jasmonate and methyl salicylate (Rodriguez-Saona et al. 2011, Pierik et al. 2014), which are used in plant defense (among other roles). An important implication of understanding plant chemical signaling is the possibility of producing VOC blends that may manipulate or interfere with interactions between plants and insects for biological control and pest management, for example, via genetic engineering (Kos et al., 2013) or more traditional approaches of semiochemical application with controlled release devices (Witzgall et al., 2010b).

It has been documented that upon infection, pathogens can alter plant VOC output to attract vectors to the host, thus potentially aiding in pathogen propagation (Eigenbrode et al., 2002; McLeod et al., 2005; Mauck et al., 2010; Davis et al., 2012; Shapiro et al., 2012). ‘Deceptive’ attraction of vectors to infected plants from which they subsequently disperse may be conducive to enhanced pathogen transmission and may be honed through natural selection of vector behavior. For example, simultaneous manipulation of both plant odorant release and nutritional quality may result in initial attraction of herbivores to nutritionally sub-optimal, pathogen-infected plants followed by subsequent dispersal to and settling on nutritionally superior, non-infected counterparts (Mauck et al., 2010; Mann et al., 2012). This mechanism may drive pathogen spread throughout a community of plant hosts and has been termed the “deceptive host phenotype hypothesis” (Mauck et al. 2010).

A notable organism in citrus plant pathology is the Asian citrus psyllid, Diaphorina citri Kuwayama, which is the insect vector of Liberibacter species pathogens among citrus hosts. D. citri is a globally invasive species and, more importantly, a vector for Liberibacter species, including Candidatus Liberibacter asiaticus (CLas) (Grafton-Cardwell et al., 2013). CLas is one of the bacterial pathogens causing huanglongbing (HLB). This disease is considered the greatest threat to citriculture worldwide (Callaway, 2008) and is propagating through South and North America. Currently, management of HLB is mainly based on chemical control of D. citri populations (Grafton-Cardwell et al., 2013). However, overuse of insecticides may negatively affect the environment and is known to cause insecticide resistance in populations of D. citri (Grafton-Cardwell et al., 2013). The behavior of D. citri appears congruent with the ‘deceptive host phenotype hypothesis’ given that D. citri are more attracted to CLas-infected plants than uninfected plants. Moreover, after initially settling on CLas-infected plants, D. citri subsequently disperse to nearby uninfected plants in search of a more nutritious host (Mann et al., 2012). Current trapping methods for D. citri rely on the use of yellow sticky traps without an associated olfactory lure. Unbaited traps capture D. citri from short distances and are marginally effective as monitoring tools (Grafton-Cardwell et al., 2013). Thus, there exists a need for an effective chemical attractant for D. citri. Development of an attractant for D. citri would not only improve monitoring, but may also allow for development of other biorational tools, such as attract-and-kill or host finding disruption formulations.

### BRIEF SUMMARY

In one aspect, the present disclosure relates to a composition containing a mixture of two or more compounds released by a citrus plant in quantities that are altered during infection with Huanglongbing disease, where the composition is an attractant for psyllids. That is, the composition is a synthetic chemical blend, which acts as a chemical lure for psyllids such as Diaphorina citri. Thus, the composition does not comprise a citrus plant with Huanglongbing disease or portions thereof. In some embodiments, the mixture includes two or more compounds selected from linalool, tridecane, 4-OH-4-Me-2-pentanone, hexacosane, 1-tetradecene, tricosane, geranial, tetradecanal, phenylacetaldehyde, methyl salicylate, cumacrene, (E)-β-ocimene, hexadecanol, and geranyl acetone. The two or more compounds include from two to fourteen of the compounds (at least two, three, four, five, six, seven, eight, nine, ten, eleven, twelve, thirteen or all fourteen of the compounds). In some embodiments, the mixture includes linalool, tricosane, geranial, phenylacetaldehyde, methyl salicylate, and (E)-β-ocimene. In some embodiments, the mixture further includes geranyl acetone. In some embodiments, in the mixture, one of linalool, methyl salicylate, geranial and combinations thereof, is present at a percent weight of at least 25% of the compounds in the mixture. In some embodiments, in the mixture, tricosane, phenylacetaldehyde, and (E)-β-ocimene are each present at a percent weight of less than 10% of the compounds in the mixture. In some embodiments, the mixture constitutes less than 5% by weight of the composition. In some embodiments that may be combined with any of the preceding embodiments, the mixture includes linalool, tridecane, 4-OH-4-Me-2-pentanone, hexacosane, 1-tetradecene, tricosane, geranial, tetradecanal, phenylacetaldehyde, methyl salicylate, cumacrene, (E)-β-ocimene, hexadecanol, and geranyl acetone. In some embodiments that may be combined with any of the preceding embodiments, when in the mixture, the ratio of linalool:4-OH-Me-2-pentanone is from 5 to 15 or about 11.81:1, the ratio of tridecane:4-OH-Me-2-pentanone is from 2 to 10 or about 6.28:1, the ratio of hexacosane:4-OH-Me-2-pentanone is from 2 to 10 or about 6.18:1, the ratio of 1-tetradecene:4-OH-Me-2-pentanone is from 2 to 10 or about 6.01:1, the ratio of tricosane:4-OH-Me-2-pentanone is from 30 to 90 or about 60.87:1, the ratio of geranial:4-OH-Me-2-pentanone is from 5 to 15 or about 9.48:1, the ratio of tetradecanal:4-OH-Me-2-pentanone is from 3 to 15 or about 7.21:1, the ratio of phenylacetaldehyde:4-OH-Me-2-pentanone is from 3 to 15 or about 8.48:1, the ratio of methyl salicylate:4-OH-Me-2-pentanone is from 5 to 20 or about 12.46:1, the ratio of cumacrene:4-OH-Me-2-pentanone is from 0.5 to 10 or about 3.50:1, the ratio of (E)-β-ocimene:4-OH-Me-2-pentanone is from 2 to 10 or about 5.08:1, the ratio of hexadecanol:4-OH-Me-2-pentanone is from 0.1 to 5 or about 1:1, and the ratio of geranyl acetone:4-OH-Me-2-pentanone is from 20 to 60 or about 43.67:1. In some embodiments that may be combined with any of the preceding embodiments, the compounds in the mixture are present at a concentration in the range of 0.01 μg/μL to 0.1 μg/μL. In some embodiments that may be combined with any of the preceding embodiments, the composition further includes a solvent. In some embodiments, the solvent is dichloromethane. In some embodiments that may be combined with any of the preceding embodiments, the composition attracts at least 50% of the psyllids in a psyllid population exposed to the composition in, for example, a laboratory setting. In some embodiments that may be combined with any of the preceding embodiments, the composition further includes an insecticide. In some embodiments that may be combined with any of the preceding embodiments, the citrus plant is Citrus sinensis L. Osbeck. In some embodiments that may be combined with any of the preceding embodiments, the psyllid is Diaphirona citri.

In another aspect, the present disclosure relates to a kit including a composition containing a mixture of two or more compounds released by a citrus plant in quantities that are altered during infection with Huanglongbing disease, where the composition is an attractant for psyllids, and where the kit is suitable for dispensing the composition. In some embodiments, the mixture includes two or more compounds selected from linalool, tridecane, 4-OH-4-Me-2-pentanone, hexacosane, 1-tetradecene, tricosane, geranial, tetradecanal, phenylacetaldehyde, methyl salicylate, cumacrene, (E)-β-ocimene, hexadecanol, and geranyl acetone. The two or more compounds include from two to fourteen of the compounds (at least two, three, four, five, six, seven, eight, nine, ten, eleven, twelve, thirteen or all fourteen of the compounds). In some embodiments, the mixture includes linalool, tricosane, geranial, phenylacetaldehyde, methyl salicylate, and (E)-β-ocimene. In some embodiments, the mixture further includes geranyl acetone. In some embodiments, in the mixture, one of linalool, methyl salicylate, geranial and combinations thereof, is present at a percent weight of at least 25% of the compounds in the mixture. In some embodiments, in the mixture, tricosane, phenylacetaldehyde, and (E)-β-ocimene are each present at a percent weight of less than 10% of the compounds in the mixture. In some embodiments, the mixture constitutes less than 5% by weight of the composition. In some embodiments that may be combined with any of the preceding embodiments, the mixture includes linalool, tridecane, 4-OH-4-Me-2-pentanone, hexacosane, 1-tetradecene, tricosane, geranial, tetradecanal, phenylacetaldehyde, methyl salicylate, cumacrene, (E)-β-ocimene, hexadecanol, and geranyl acetone. In some embodiments that may be combined with any of the preceding embodiments, when in the mixture, the ratio of linalool:4-OH-Me-2-pentanone is from 5 to 15 or about 11.81:1, the ratio of tridecane:4-OH-Me-2-pentanone is from 2 to 10 or about 6.28:1, the ratio of hexacosane:4-OH-Me-2-pentanone is from 2 to 10 or about 6.18:1, the ratio of 1-tetradecene:4-OH-Me-2-pentanone is from 2 to 10 or about 6.01:1, the ratio of tricosane:4-OH-Me-2-pentanone is from 30 to 90 or about 60.87:1, the ratio of geranial:4-OH-Me-2-pentanone is from 5 to 15 or about 9.48:1, the ratio of tetradecanal:4-OH-Me-2-pentanone is from 3 to 15 or about 7.21:1, the ratio of phenylacetaldehyde:4-OH-Me-2-pentanone is from 3 to 15 or about 8.48:1, the ratio of methyl salicylate:4-OH-Me-2-pentanone is from 5 to 20 or about 12.46:1, the ratio of cumacrene:4-OH-Me-2-pentanone is from 0.5 to 10 or about 3.50:1, the ratio of (E)-β-ocimene:4-OH-Me-2-pentanone is from 2 to 10 or about 5.08:1, the ratio of hexadecanol:4-OH-Me-2-pentanone is from 0.1 to 5 or about 1:1, and the ratio of geranyl acetone:4-OH-Me-2-pentanone is from 20 to 60 or about 43.67:1. In some embodiments that may be combined with any of the preceding embodiments, the compounds in the mixture are present at a concentration in the range of 0.01 μg/μL to 0.1 μg/μL. In some embodiments that may be combined with any of the preceding embodiments, the composition further includes a solvent. In some embodiments, the solvent is dichloromethane. In some embodiments that may be combined with any of the preceding embodiments, the composition attracts at least 50% of the psyllids in a psyllid population exposed to the composition in, for example, a laboratory setting. In some embodiments that may be combined with any of the preceding embodiments, the composition further includes an insecticide. In some embodiments that may be combined with any of the preceding embodiments, the citrus plant is Citrus sinensis L. Osbeck. In some embodiments that may be combined with any of the preceding embodiments, the psyllid is Diaphirona citri. In some embodiments that may be combined with any of the preceding embodiments, the composition is dispensed as a liquid. In some embodiments that may be combined with any of the preceding embodiments, the composition is dispensed as an aerosol.

In another aspect, the present disclosure relates to a method of attracting a psyllid, the method including: a) providing an environment including psyllids, and b) contacting the environment with a composition containing a mixture of two or more compounds released by a citrus plant in quantities that are altered during infection with Huanglongbing disease, where the composition is an attractant for psyllids, and where said composition is present at a source location, and where a psyllid is attracted to said composition. In another aspect, the present disclosure relates to a method of monitoring psyllid infestation, the method including: a) placing a composition containing a mixture of two or more compounds released by a citrus plant in quantities that are altered during infection with Huanglongbing disease in a citrus orchard at a source location, and b) monitoring contact of the composition by a psyllid, where the contact is indicative of psyllid infestation. In some embodiments, the mixture includes two or more compounds selected from linalool, tridecane, 4-OH-4-Me-2-pentanone, hexacosane, 1-tetradecene, tricosane, geranial, tetradecanal, phenylacetaldehyde, methyl salicylate, cumacrene, (E)-β-ocimene, hexadecanol, and geranyl acetone. The two or more compounds include from two to fourteen of the compounds (at least two, three, four, five, six, seven, eight, nine, ten, eleven, twelve, thirteen or all fourteen of the compounds). In some embodiments, the mixture includes linalool, tricosane, geranial, phenylacetaldehyde, methyl salicylate, and (E)-β-ocimene. In some embodiments, the mixture further includes geranyl acetone. In some embodiments, in the mixture, one of linalool, methyl salicylate, geranial and combinations thereof, is present at a percent weight of at least 25% of the compounds in the mixture. In some embodiments, in the mixture, tricosane, phenylacetaldehyde, and (E)-β-ocimene are each present at a percent weight of less than 10% of the compounds in the mixture. In some embodiments, the mixture constitutes less than 5% by weight of the composition. In some embodiments that may be combined with any of the preceding embodiments, the mixture includes linalool, tridecane, 4-OH-4-Me-2-pentanone, hexacosane, 1-tetradecene, tricosane, geranial, tetradecanal, phenylacetaldehyde, methyl salicylate, cumacrene, (E)-β-ocimene, hexadecanol, and geranyl acetone. In some embodiments that may be combined with any of the preceding embodiments, when in the mixture, the ratio of linalool:4-OH-Me-2-pentanone is from 5 to 15 or about 11.81:1, the ratio of tridecane:4-OH-Me-2-pentanone is from 2 to 10 or about 6.28:1, the ratio of hexacosane:4-OH-Me-2-pentanone is from 2 to 10 or about 6.18:1, the ratio of 1-tetradecene:4-OH-Me-2-pentanone is from 2 to 10 or about 6.01:1, the ratio of tricosane:4-OH-Me-2-pentanone is from 30 to 90 or about 60.87:1, the ratio of geranial:4-OH-Me-2-pentanone is from 5 to 15 or about 9.48:1, the ratio of tetradecanal:4-OH-Me-2-pentanone is from 3 to 15 or about 7.21:1, the ratio of phenylacetaldehyde:4-OH-Me-2-pentanone is from 3 to 15 or about 8.48:1, the ratio of methyl salicylate:4-OH-Me-2-pentanone is from 5 to 20 or about 12.46:1, the ratio of cumacrene:4-OH-Me-2-pentanone is from 0.5 to 10 or about 3.50:1, the ratio of (E)-β-ocimene:4-OH-Me-2-pentanone is from 2 to 10 or about 5.08:1, the ratio of hexadecanol:4-OH-Me-2-pentanone is from 0.1 to 5 or about 1:1, and the ratio of geranyl acetone:4-OH-Me-2-pentanone is from 20 to 60 or about 43.67:1. In some embodiments that may be combined with any of the preceding embodiments, the compounds in the mixture are present at a concentration in the range of 0.01 μg/μL to 0.1 μg/μL. In some embodiments that may be combined with any of the preceding embodiments, the composition further includes a solvent. In some embodiments, the solvent is dichloromethane. In some embodiments that may be combined with any of the preceding embodiments, the composition attracts at least 50% of the psyllids in a psyllid population exposed to the composition in, for example, a laboratory setting. In some embodiments that may be combined with any of the preceding embodiments, the composition further includes an insecticide. In some embodiments that may be combined with any of the preceding embodiments, the citrus plant is Citrus sinensis L. Osbeck. In some embodiments that may be combined with any of the preceding embodiments, the psyllid is Diaphirona citri. In some embodiments that may be combined with any of the preceding embodiments, the composition is present at a stationary source location. In some embodiments that may be combined with any of the preceding embodiments, the method further includes a step of monitoring the source location for contact with a psyllid. In some embodiments, the monitoring occurs over a time interval. In some embodiments, the method further includes a step of monitoring changes in psyllid contact with the source location over the time interval. In some embodiments, changes in psyllid contact are changes in the number of psyllids contacting the composition or the duration of psyllid contact with the composition. In some embodiments that may be combined with any of the preceding embodiments, the method further includes a step of terminating a psyllid that contacts the composition. In some embodiments that may be combined with any of the preceding embodiments, the environment is an open or a closed environment. In some embodiments that may be combined with any of the preceding embodiments, the environment is a citrus orchard. In some embodiments that may be combined with any of the preceding embodiments, the citrus orchard is an orange tree orchard. In some embodiments that may be combined with any of the preceding embodiments, the psyllid is Diaphirona citri.

### DESCRIPTION OF THE FIGURES

The patent or application file contains at least one drawing executed in color. Copies of this patent or patent application publication with color drawings will be provided by the office upon request and payment of the necessary fee.

FIG. 1 illustrates volatile biomarker compounds differentially expressed in uninfected plants and plants infected with C. liberibacter that are common throughout the independent studies. The listed compounds were detected using Twister GC/MS.

FIG. 2 illustrates a schematic diagram of the behavioral assay of Diaphirona citri response to the ‘HLB’ (infected) blend vs. citrus plant volatiles.

FIG. 3 illustrates the Attenu assay fluorescence plot for binding of an equimolar mixture of 14 HLB biomarkers compounds with a chemosensory protein DcOBP1 at 4 uM. A mixture of the 14 compounds was made in which each compound was present at 10 μM. Two replicate runs are shown for experiments both with and without the mix of semiochemicals.

FIG. 4 illustrates the Attenu assay fluorescence plots for mixtures representing semiochemical emissions of 14 HLB biomarkers compounds with the Diaphorina citri chemosensory proteins for DcOBP1 at 4 μM. Representative error bars show one standard deviation for the data generated for 4 μM DcOBP1 protein with positive control (indole). Protein=protein at corresponding concentration. Dye=Fluorescent dye without protein or ligand. Healthy+=uninfected mixture with tricosane. Healthy−=uninfected mixture without tricosane. HLB+=HLB mixture with tricosane. HLB−=HLB mixture without tricosane. Positive Control=5 μM known ligand (indole) for corresponding protein.

FIG. 5 illustrates the Attenu assay fluorescence plots for mixtures representing semiochemical emissions of 14 HLB biomarkers compounds with the Diaphorina citri chemosensory proteins for DcSAP1 at 10 μM. Protein=protein at corresponding concentration. Dye=Fluorescent dye without protein or ligand. Healthy+=uninfected mixture with tricosane. Healthy−=uninfected mixture without tricosane. HLB+=HLB mixture with tricosane. HLB−=HLB mixture without tricosane. Positive Control=5 μM known ligand (indole) for corresponding protein.

FIG. 6 illustrates the Attenu assay fluorescence plots for mixtures representing semiochemical emissions of 14 HLB biomarkers compounds with the Diaphorina citri chemosensory proteins for DcSAP2 at 10 μM. Protein=protein at corresponding concentration. Dye=Fluorescent dye without protein or ligand. Healthy+=uninfected mixture with tricosane. Healthy−=uninfected mixture without tricosane. HLB+=HLB mixture with tricosane. HLB−=HLB mixture without tricosane. Positive Control=5 μM known ligand (indole) for corresponding protein.

FIG. 7 illustrates the Attenu assay fluorescence plots for mixtures representing semiochemical emissions of 14 HLB biomarkers compounds with the Diaphorina citri chemosensory proteins for DcSAP3 at 10 μM. Representative error bars show one standard deviation for the data generated for 10 μM DcSAP3 protein with positive control (indole). Protein=protein at corresponding concentration. Dye=Fluorescent dye without protein or ligand. Healthy+=uninfected mixture with tricosane. Healthy−=uninfected mixture without tricosane. HLB+=HLB mixture with tricosane. HLB−=HLB mixture without tricosane. Positive Control=5 μM known ligand (indole) for corresponding protein.

FIG. 8 illustrates the Attenu assay fluorescence plots for mixtures representing semiochemical emissions of 14 HLB biomarkers compounds with the Diaphorina citri chemosensory proteins for DcSAP4 at 10 μM. Protein=protein at corresponding concentration. Dye=Fluorescent dye without protein or ligand. Healthy+=uninfected mixture with tricosane. Healthy−=uninfected mixture without tricosane. HLB+=HLB mixture with tricosane. HLB−=HLB mixture without tricosane. Positive Control=5 μM known ligand (indole) for corresponding protein.

FIG. 9 illustrates the responses of Diaphorina citri when presented with volatiles emanating from ‘Healthy’ (uninfected), “HLB” (infected) synthetic blends, from uninfected healthy citrus plant volatiles, or from solvent controls in various comparisons to each other (comparisons with the respective treatments on the indicated side of the bar). NR: Non responder. Stars indicate significant difference between treatments. *: P<0.05, **: P<0.01.

FIG. 10A-FIG. 10E illustrates average values (±SE) of the areas under the fluorescence curves for the Attenu assays. A reduction of the fluorescence compared to the average fluorescence of the protein alone (dotted lines) indicates an interaction between the protein and the mixture tested. (FIG. 10A) DcOBP1, (FIG. 10B) DcSAP1, (FIG. 10C) DcSAP2, (FIG. 10D) DcSAP3, (FIG. 10E) DcSAP4. (Healthy+), uninfected mixture with tricosane. (Healthy−), uninfected mixture without tricosane; (HLB+), HLB mixture with tricosane; (HLB−), HLB mixture without tricosane. Total concentration of semiochemicals is 10 μM. ***P<0.001, **P<0.01, *P<0.05, ^{ø}P<0.1. RFU²: Relative fluorescence units. Note differences in y-axes scales.

FIG. 11A-FIG. 11B illustrate psyllid attraction assays using synthetic chemical blends. FIG. 11A illustrates the results of principal component analysis on the distribution of odor compounds from different types of citrus trees, ranging from healthy to severe HLB infection. FIG. 11B illustrates the responses of Diaphorina citri when presented with volatiles emanating from ‘Healthy’ (uninfected), “HLB” (infected) synthetic blends, from uninfected healthy citrus plant volatiles, or from solvent controls in various comparisons to each other (comparisons with the respective treatments on the indicated side of the bar). NR: Non responder. Stars indicate significant difference between treatments. *: P<0.05, **: P<0.01.

FIG. 12 illustrates the results of different chemical blend formulations on the ability to attract ACP. On the left-hand panel, compounds 1-8 (top row, X-axis) are as follows: 1: (E)-Beta-Ocimene; 2: Tricosane; 3: Phenylacetaldehyde; 4: Linalool; 5:1-Tetradecene; 6: Geranyl Acetone; 7: Geranial; 8: Methyl Salicylate. White open circles are indicative that the listed compound is absent in the formulation, whereas closed black circles are indicative that the listed compound is present in the formulation. The behavioral response of ACP to each formulation is shown in the right-hand panel.

FIG. 13 illustrates the results of different chemical blend formulations on the ability to attract ACP. On the left-hand panel, compounds 1-7 (top row, X-axis) are as follows: 1: Tricosane; 2 Geranial; 3: Methyl Salicylate; 4: Geranyl Acetone; 5: Linalool; 6: Phenylacetaldehyde; 7: (E)-Beta-Ocimene. The concentrations of each compound, shown as the percentage of the total active compounds in the formulation, are shown. The behavioral response of ACP to each formulation is shown in the right-hand panel.

FIG. 14 illustrates the results of different chemical blend formulations on the ability to attract ACP. On the left-hand panel, compounds 1-7 (top row, X-axis) are as follows: 1: Tricosane; 2 Geranial; 3: Methyl Salicylate; 4: Geranyl Acetone; 5: Linalool; 6: Phenylacetaldehyde; 7: (E)-Beta-Ocimene. The concentrations of each compound, shown as the percentage of the total active compounds in the formulation, are shown. The behavioral response of ACP to each formulation is shown in the right-hand panel.

FIG. 15 illustrates the compositions of two different synthetic chemical lures of Asian citrus psyllid that were tested in field conditions.

FIG. 16A-FIG. 16B illustrate various aspects of testing synthetic chemical blends to lure ACP in field trial settings. FIG. 16A illustrates the trap device used to capture psyllids in the field trials. FIG. 16B illustrates the results of the tested synthetic chemical blends with respect to their ability to lure ACP.

FIG. 17 illustrates the results of tested synthetic chemical blends that also contain an insecticide with respect to their ability to lure ACP.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

The present disclosure relates to chemical lures for Diaphorina citri (Asian citrus psyllid) and methods of using these chemical lures.

The present disclosure is based, at least in part, on Applicant's development of a synthetic chemical blend which mimics the blend of volatiles released by citrus trees during infection with Huanglongbing disease. Applicant's synthetic blend was able to act as a lure and attracted Diaphorina citri (Asian citrus psyllid). This synthetic blend may be used in volatile dispersion kits that disperse the volatilized synthetic blend into an environment to attract psyllids such as Diaphorina citri. Such volatile dispersion kits containing the synthetic blend may be used to monitor psyllid activity or in bait-and-kill stations to control psyllid populations.

Accordingly, the present disclosure provides compositions containing synthetic chemical blends for attracting psyllids, kits for dispensing these compositions into an environment, and methods of using these compositions to attract psyllids.

Unless defined otherwise, all scientific and technical terms are understood to have the same meaning as commonly used in the art to which they pertain. For the purpose of the present disclosure, the following terms are defined.

The use of the terms “a,” “an,” and “the,” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The use of the term “about” with regard to a numerical value is to be construed as including a value within the range of standard experimental or mechanical error. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the embodiments of the disclosure and does not pose a limitation on the scope of the disclosure unless otherwise claimed. No language in the specification should be construed as indicating any non-claimed element as essential to the practice of the embodiments of the disclosure.

### Compositions of the Disclosure

The present disclosure relates to compositions containing a mixture of e.g. one or more or two or more compounds released by a citrus plant in quantities that are altered during infection with Huanglongbing disease, where the composition is an attractant for psyllids. In some embodiments, compositions of the disclosure contain one or more active compounds which constitute a synthetic chemical blend for attracting psyllids. Active compounds in the compositions may include, for example, one or more compounds selected from linalool, tridecane, 4-OH-4-Me-2-pentanone, hexacosane, 1-tetradecene, tricosane, geranial, tetradecanal, phenylacetaldehyde, methyl salicylate, cumacrene, (E)-β-ocimene, hexadecanol, and geranyl acetone.

Compositions of the present disclosure may include, for example, one or more, two or more, three or more, four or more, five or more, six or more, seven or more, eight or more, nine or more, ten or more, eleven or more, twelve or more, or thirteen or more compounds selected from the compounds linalool, tridecane, 4-OH-4-Me-2-pentanone, hexacosane, 1-tetradecene, tricosane, geranial, tetradecanal, phenylacetaldehyde, methyl salicylate, cumacrene, (E)-β-ocimene, hexadecanol, and geranyl acetone. In some embodiments, compositions of the present disclosure contain each of linalool, tridecane, 4-OH-4-Me-2-pentanone, hexacosane, 1-tetradecene, tricosane, geranial, tetradecanal, phenylacetaldehyde, methyl salicylate, cumacrene, (E)-β-ocimene, hexadecanol, and geranyl acetone.

In some embodiments, compositions of the present disclosure contain linalool, tricosane, geranial, phenylacetaldehyde, methyl salicylate, and (E)-β-ocimene. In some embodiments, these compositions also contain geranyl acetone.

Active compounds in the compositions of the present disclosure such as, for example, linalool, tridecane, 4-OH-4-Me-2-pentanone, hexacosane, 1-tetradecene, tricosane, geranial, tetradecanal, phenylacetaldehyde, methyl salicylate, cumacrene, (E)-β-ocimene, hexadecanol, and/or geranyl acetone, may be present in the compositions at, for example, about 0.001% to about 0.005%, about 0.005% to about 0.01%, about 0.01% to about 0.05%, about 0.05% to about 0.1%, about 0.1% to about 0.5%, about 0.5% to about 1%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% or more of the total dry weight of the synthetic chemical blend in the composition.

Active compounds in the compositions of the present disclosure such as, for example, linalool, tridecane, 4-OH-4-Me-2-pentanone, hexacosane, 1-tetradecene, tricosane, geranial, tetradecanal, phenylacetaldehyde, methyl salicylate, cumacrene, (E)-β-ocimene, hexadecanol, and/or geranyl acetone, may be present in the compositions at, for example, about 0.001% to about 0.005%, about 0.005% to about 0.01%, about 0.01% to about 0.05%, about 0.05% to about 0.1%, about 0.1% to about 0.5%, about 0.05% to about 1%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% or more of the total dry weight of the composition.

In embodiments where compositions of the present disclosure contain linalool, this compound may be present in the compositions at, for example, about 0.001% to about 0.005%, about 0.005% to about 0.01%, about 0.01% to about 0.05%, about 0.05% to about 0.1%, about 0.1% to about 0.5%, about 0.05% to about 1%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% or more of the total dry weight of the active components in the composition.

In embodiments where compositions of the present disclosure contain tridecane, this compound may be present in the compositions at, for example, about 0.001% to about 0.005%, about 0.005% to about 0.01%, about 0.01% to about 0.05%, about 0.05% to about 0.1%, about 0.1% to about 0.5%, about 0.05% to about 1%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% or more of the total dry weight of the active components in the composition.

In embodiments where compositions of the present disclosure contain 4-OH-4-Me-2-pentanone, this compound may be present in the compositions at, for example, about 0.001% to about 0.005%, about 0.005% to about 0.01%, about 0.01% to about 0.05%, about 0.05% to about 0.1%, about 0.1% to about 0.5%, about 0.05% to about 1%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% or more of the total dry weight of the active components in the composition.

In embodiments where compositions of the present disclosure contain hexacosane, this compound may be present in the compositions at, for example, about 0.001% to about 0.005%, about 0.005% to about 0.01%, about 0.01% to about 0.05%, about 0.05% to about 0.1%, about 0.1% to about 0.5%, about 0.05% to about 1%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% or more of the total dry weight of the active components in the composition.

In embodiments where compositions of the present disclosure contain 1-tetradecene, this compound may be present in the compositions at, for example, about 0.001% to about 0.005%, about 0.005% to about 0.01%, about 0.01% to about 0.05%, about 0.05% to about 0.1%, about 0.1% to about 0.5%, about 0.05% to about 1%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% or more of the total dry weight of the active components in the composition.

In embodiments where compositions of the present disclosure contain tricosane, this compound may be present in the compositions at, for example, about 0.001% to about 0.005%, about 0.005% to about 0.01%, about 0.01% to about 0.05%, about 0.05% to about 0.1%, about 0.1% to about 0.5%, about 0.05% to about 1%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% or more of the total dry weight of the active components in the composition.

In embodiments where compositions of the present disclosure contain geranial, this compound may be present in the compositions at, for example, about 0.001% to about 0.005%, about 0.005% to about 0.01%, about 0.01% to about 0.05%, about 0.05% to about 0.1%, about 0.1% to about 0.5%, about 0.05% to about 1%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% or more of the total dry weight of the active components in the composition.

In embodiments where compositions of the present disclosure contain tetradecanal, this compound may be present in the compositions at, for example, about 0.001% to about 0.005%, about 0.005% to about 0.01%, about 0.01% to about 0.05%, about 0.05% to about 0.1%, about 0.1% to about 0.5%, about 0.05% to about 1%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% or more of the total dry weight of the active components in the composition.

In embodiments where compositions of the present disclosure contain phenylacetaldehyde, this compound may be present in the compositions at, for example, about 0.001% to about 0.005%, about 0.005% to about 0.01%, about 0.01% to about 0.05%, about 0.05% to about 0.1%, about 0.1% to about 0.5%, about 0.05% to about 1%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% or more of the total dry weight of the active components in the composition.

In embodiments where compositions of the present disclosure contain methyl salicylate, this compound may be present in the compositions at, for example, about 0.001% to about 0.005%, about 0.005% to about 0.01%, about 0.01% to about 0.05%, about 0.05% to about 0.1%, about 0.1% to about 0.5%, about 0.05% to about 1%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% or more of the total dry weight of the active components in the composition.

In embodiments where compositions of the present disclosure contain cumacrene, this compound may be present in the compositions at, for example, about 0.001% to about 0.005%, about 0.005% to about 0.01%, about 0.01% to about 0.05%, about 0.05% to about 0.1%, about 0.1% to about 0.5%, about 0.05% to about 1%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% or more of the total dry weight of the active components in the composition.

In embodiments where compositions of the present disclosure contain (E)-β-ocimene, this compound may be present in the compositions at, for example, about 0.001% to about 0.005%, about 0.005% to about 0.01%, about 0.01% to about 0.05%, about 0.05% to about 0.1%, about 0.1% to about 0.5%, about 0.05% to about 1%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% or more of the total dry weight of the active components in the composition.

In embodiments where compositions of the present disclosure contain hexadecanol, this compound may be present in the compositions at, for example, about 0.001% to about 0.005%, about 0.005% to about 0.01%, about 0.01% to about 0.05%, about 0.05% to about 0.1%, about 0.1% to about 0.5%, about 0.05% to about 1%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% or more of the total dry weight of the active components in the composition.

In embodiments where compositions of the present disclosure contain geranyl acetone, this compound may be present in the compositions at, for example, about 0.001% to about 0.005%, about 0.005% to about 0.01%, about 0.01% to about 0.05%, about 0.05% to about 0.1%, about 0.1% to about 0.5%, about 0.05% to about 1%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% or more of the total dry weight of the active components in the composition.

Active compounds in the compositions of the present disclosure such as, for example, one or more of linalool, tridecane, 4-OH-4-Me-2-pentanone, hexacosane, 1-tetradecene, tricosane, geranial, tetradecanal, phenylacetaldehyde, methyl salicylate, cumacrene, (E)-β-ocimene, hexadecanol, and/or geranyl acetone, may be present in the compositions at various concentrations relative to other compounds in the composition. For example, the concentrations of the active compounds in the compositions in the present disclosure may be relative to the concentration of 4-OH-Me-2-pentanone in the composition. For example, when linalool is present in the composition, the ratio of linalool:4-OH-Me-2-pentanone in the composition may be about 5 to about 15:1. When tridecane is present in the composition, the ratio of tridecane:4-OH-Me-2-pentanone in the composition may be, for example, about 2 to about 10:1. When hexacosane is present in the composition, the ratio of hexacosane:4-OH-Me-2-pentanone in the composition may be, for example, about 2 to about 10:1. When 1-tetradecene is present in the composition, the ratio of 1-tetradecene:4-OH-Me-2-pentanone in the composition may be, for example, about 2 to about 10:1. When tricosane is present in the composition, the ratio of tricosane:4-OH-Me-2-pentanone in the composition may be, for example, about 30 to about 90:1. When geranial is present in the composition, the ratio of geranial:4-OH-Me-2-pentanone in the composition may be, for example, about 5 to about 15:1. When tetradecanal is present in the composition, the ratio of tetradecanal:4-OH-Me-2-pentanone in the composition may be, for example, about 3 to about 15:1. When phenylacetaldehyde is present in the composition, the ratio of phenylacetaldehyde:4-OH-Me-2-pentanone in the composition may be, for example, about 3 to about 15:1. When methyl salicylate is present in the composition, the ratio of methyl salicylate:4-OH-Me-2-pentanone in the composition may be, for example, about 5 to about 20:1. When cumacrene is present in the composition, the ratio of cumacrene:4-OH-Me-2-pentanone in the composition may be, for example, about 0.5 to about 10:1. When (E)-β-ocimene is present in the composition, the ratio of (E)-β-ocimene:4-OH-Me-2-pentanone in the composition may be, for example, about 2 to about 10:1. When hexadecanol is present in the composition, the ratio of hexadecanol:4-OH-Me-2-pentanone in the composition may be, for example, about 0.1 to about 5:1. When geranyl acetone is present in the composition, the ratio of geranyl acetone:4-OH-Me-2-pentanone in the composition may be, for example, about 20 to about 60:1.

Active compounds in the compositions of the present disclosure such as, for example, one or more of linalool, tridecane, 4-OH-4-Me-2-pentanone, hexacosane, 1-tetradecene, tricosane, geranial, tetradecanal, phenylacetaldehyde, methyl salicylate, cumacrene, (E)-β-ocimene, hexadecanol, and/or geranyl acetone, may be present in the compositions at various concentrations. The total concentration of active compounds in the compositions of the present disclosure may be, for example, about 5 μM, about 6 μM, about 7 μM, about 8 μM, about 9 μM, about 10 μM, about 11 μM, about 12 μM, about 13 μM, about 14 μM, or about 15 μM or higher. The active compounds may be present in the compositions of the present disclosure at a concentration of, for example, about 0.001 to about 0.005 μg/μL, about 0.005 to about 0.01 μg/μL, about 0.01 to about 0.05 μg/μL, about 0.05 to about 0.1 μg/μL, about 0.1 to about 0.5 μg/μL, or about 0.5 to about 1 μg/μL or higher concentration.

Compositions of the present disclosure may also include an insecticide. Various insecticides may be used such as, for example, Spinosad. One of skill in the art would readily recognize additional insecticides that may be used in the methods and compositions of the present disclosure. Insecticides may be present in compositions of the present disclosure at, for example, about 0.001% to about 0.005%, about 0.005% to about 0.01%, about 0.01% to about 0.05%, about 0.05% to about 0.1%, about 0.05% to about 0.2%, about 0.05% to about 0.3%, about 0.05% to about 0.4%, about 0.05% to about 0.5%, about 0.1% to about 0.5%, about 0.1% to about 1%, about 0.2% to about 1%, about 0.3% to about 1%, about 0.4% to about 1%, about 1% to about 2%, about 2% to about 5%, or about 5% or more of the total weight of the composition, or the total weight of the active components in the composition.

### Kits of the Disclosure

The present disclosure relates to kits containing a composition containing a mixture of e.g. one or more or two or more compounds released by a citrus plant in quantities that are altered during infection with Huanglongbing disease, where the composition is an attractant for psyllids, and where the kit is suitable for dispensing the composition. Kits of the present disclosure contain one or more compounds of the present disclosure such as, for example, one or more compounds selected from linalool, tridecane, 4-OH-4-Me-2-pentanone, hexacosane, 1-tetradecene, tricosane, geranial, tetradecanal, phenylacetaldehyde, methyl salicylate, cumacrene, (E)-β-ocimene, hexadecanol, and geranyl acetone.

Kits of the present disclosure are suitable for dispensing compositions of the present disclosure into an environment. In some embodiments, compositions of the present disclosure contain a solvent such as, for example, dichloromethane, in addition to the active compounds. In some embodiments, chemical standards of the active compounds are mixed into a synthetic chemical blend, where the composition is substantially composed of the synthetic chemical blend.

The kits may dispense compositions of the present disclosure into an environment in various concentrations and over various time intervals. In some embodiments, the kit dispenses a composition having a concentration of active compounds in the range of about 0.01 μg/μL to about 0.1 μg/μL. The kit may dispense the compositions into an environment at various times or over various time intervals. For example, the kit may dispense a composition of the present disclosure once a day, or a kit may dispense a composition of the present disclosure several times over the course of a day such as, for example, several times over the course of one or more hours in a day.

Kits of the present disclosure for dispensing compositions of the present disclosure are different from plants which naturally emit volatile chemicals. A kit of the present disclosure is a man-made apparatus which dispenses compositions of the present disclosure into an environment. Various kits which may dispense a composition of the present disclosure into an environment are described herein and will be readily apparent to one of skill in the art.

### Methods of the Disclosure

The present disclosure relates to methods of attracting a psyllid, the method including: a) providing an environment including psyllids, and b) contacting the environment with a composition containing a mixture of e.g. one or more or two or more compounds released by a citrus plant in quantities that are altered during infection with Huanglongbing disease, where the composition is an attractant for psyllids, and where said composition is present at a source location, and where a psyllid is attracted to said composition.

One of skill in the art would readily recognize appropriate environments for use in the methods of the present disclosure in view of the guidance provided herein. For example, the environment may be a greenhouse, a closed laboratory setting, an orchard, a farm, etc. Similarly, one of skill in the art would readily recognize appropriate source locations for use in the methods of the present disclosure in view of the guidance provided herein. One of skill in the art would readily understand that the source location is a location within the psyllid-containing environment and is the source of the volatile chemical compounds dispersing from compositions of the present disclosure. The source location may be, for example, a tree branch which has placed on it a kit dispensing a composition of the present disclosure. The source location may be, for example, a kit for dispensing a composition of the present disclosure that is mounted on a man-made apparatus in an orchard or farm.

As described above, the compositions for use in the methods of the present disclosure may contain one or more compounds of the present disclosure such as, for example, one or more compounds selected from linalool, tridecane, 4-OH-4-Me-2-pentanone, hexacosane, 1-tetradecene, tricosane, geranial, tetradecanal, phenylacetaldehyde, methyl salicylate, cumacrene, (E)-β-ocimene, hexadecanol, and geranyl acetone.

After the compounds in the composition are contacted with the environment, a psyllid will be attracted to the compounds and may contact the source location. Because the compositions contain certain compounds which are volatile chemicals, these volatiles will diffuse into the environment following dispersion. Accordingly, psyllid contact with the source location includes, for example, direct contact with the source location, or indirect contact with the source location via interaction with the diffused volatiles that originated from the source location.

The source location may be monitored for contact with a psyllid, and this monitoring may occur over a time interval. Monitoring the source location may be used to inform changes in psyllid population in the environment contacted with a composition of the present disclosure.

The methods of the present disclosure, in addition to monitoring psyllid populations, may also be used to remove psyllids from the environment. For example, a psyllid that contacts the source location may be terminated, which may help to reduce the spread of plant pathogens by the psyllid.

### EXAMPLES

To better facilitate an understanding of the embodiments of the disclosure, the following examples are presented. The following examples are merely illustrative and are not meant to limit any embodiments of the present disclosure in any way.

### Example 1: Synthetic Blends of Volatile, Phytopathogen-Induced Odorants can be Used to Manipulate Vector Behavior

The Example demonstrates the development of a chemical lure for Diaphorina citri, commonly known as the Asian citrus psyllid. Volatile organic compounds (VOCs) are emitted from all plants and these VOCs are important means of communication between plants and insects. It has been documented that pathogen infections alter VOC profiles rendering infected plants more attractive to specific vectors transmitting these pathogens than uninfected plants, thus potentially aiding in pathogen propagation. Mimicking these chemical cues might enable insect attraction away from the plant or disruption of host finding behavior of the vector. However, the practical implications have not been fully explored.

In this study, it was investigated whether it is possible to exploit the “deceptive host phenotype hypothesis” for practical application to attract insect vectors by identifying and mimicking the chemical cues produced by pathogen-infected trees. This study employed citrus (host), Diaphorina citri (vector), and huanglongbing (HLB)(disease) as a model host-vector-disease system because HLB threatens citrus production worldwide and is similar to other critical diseases of food crops, such as Zebra Chip affecting potato. Applicants formulated a synthetic chemical blend using selected HLB-specific biomarker compounds, and tested the blend with the Attenu assay system for chemosensory proteins. The Attenu assay system is a procedure that identifies interactions between insect chemosensory proteins and their ligands. It was found that an equimolar mixture of compounds in the volatile profile of HLB-infected citrus bound chemosensory proteins. Further investigation of this blend in laboratory behavioral assays resulted in development of a synthetic lure that was more attractive to D. citri than natural citrus tree volatiles. This strategy provides a new route to produce chemical lures for vector population control for a variety of plant and/or animal systems and it may result in the development of a practical lure for monitoring vectors of disease, such as D. citri.

### Materials and Methods

GC-MS and qPCR Analyses

Volatiles were collected from Hamlin sweet orange (Citrus sinensis L. Osbeck) trees using polydimethylsiloxane (PDMS)-based Twister™ (GERSTEL, Inc.) sorbent beads as described by Aksenov et al. (2014). Trees were sampled at the University of Florida Citrus Research and Education Center (CREC), Lake Alfred, Fla., USA. Infected and uninfected trees were selected by professional scouts and the infection status of the trees was confirmed using quantitative qPCR. The qPCR analysis for DNA extracts was conducted according to standard methods for detection of the CLas bacterium (Pelz-Stelinski et al., 2010). Four primers and one Taqman probe empirically designed based on 16S sequences of CLas species were used (Pelz-Stelinski et al., 2010). The reverse primer (HLBr) used is specific to the genus Liberibacter and recognizes all three species within the genus.

Three independent studies were conducted to account for weather and seasonal variations in VOC production: winter (Trial 1), spring (Trial 2) and fall (Trial 3). The detailed description of the experimental protocol is provided in (Aksenov et al. 2014). The volatile compounds captured by the Twister™ sorbent were thermally desorbed and analyzed by gas chromatography-mass spectrometry (GC-MS) as described in (Skogerson et al., 2011). Briefly, a 6890 GC (Agilent Technologies, Santa Clara, Calif.) was used, which was equipped with a thermal desorption unit (TDU, GERSTEL, Inc., Muehlheim, Germany) with a cryo-cooled injection system inlet (CIS4, GERSTEL, Inc.), and interfaced to the Pegasus IV time-of-flight mass spectrometer (LECO, St. Joseph, Mich.). The volatiles trapped using Twisters were thermally desorbed in the TDU in splitless mode. The desorbed analytes were cryofocused in the CIS4 inlet with liquid nitrogen (−120° C.), heated from −120° C. to 260° C. and were analyzed on a Rtx-5SilMS column with a 10 m integrated guard column (95% dimethyl/5% diphenyl polysiloxane film; 30 m×0.25 mm (inside diameter)×0.25 μm d_{f} (Restek, Bellefonte, Pa.)). The GC oven temperature program was set as follows: initial temperature of 45° C. with a 2 min hold, followed by a 20° C./min ramp up to 300° C. with a 2 min hold, and thereafter a 20° C./min ramp up to 330° C. with a 0.5 min hold with a constant 1 mL/min flow of the carrier gas (99.9% He). Mass spectra were acquired at 25 spectra/sec with a mass range of 35-500 m/z, with the detector voltage set at 1800 V and the ionization energy at 70 eV. Raw GC-MS data were pre-processed by Leco ChromaTOF software. The compounds were identified based on similarity of mass spectra and retention indices to that of the corresponding chemical standards (Skogerson et al., 2011). The chemical standards of the selected compounds were then purchased from Sigma-Aldrich (St. Louis, Mo. USA) and TCI America (Portland, Oreg. USA) for development of a synthetic blend. If a compound did not meet the similarity score threshold (Skogerson et al., 2011), it was presumed unidentified and assigned a database entry number. The list of compounds, both identified and unidentified and their corresponding abundances, was generated for each sample. In the generated tables of compounds, every peak was normalized against the sum of the peak intensities.

Selection of Chemoattractant Compounds

The comprehensive lists of compounds produced by uninfected and infected sweet orange trees reported by Aksenov et al. (2014) were analyzed to select subsets of compounds for the use as chemoattractants in this investigation. In order to reduce the large number of compounds that differed between uninfected and infected trees to a single subset that was universal across varying stages of infection, infection subgroups with various severities of symptoms were combined into one ‘HLB’ group. The student's t-test was then performed for each individual study with the alpha value set to 0.1.

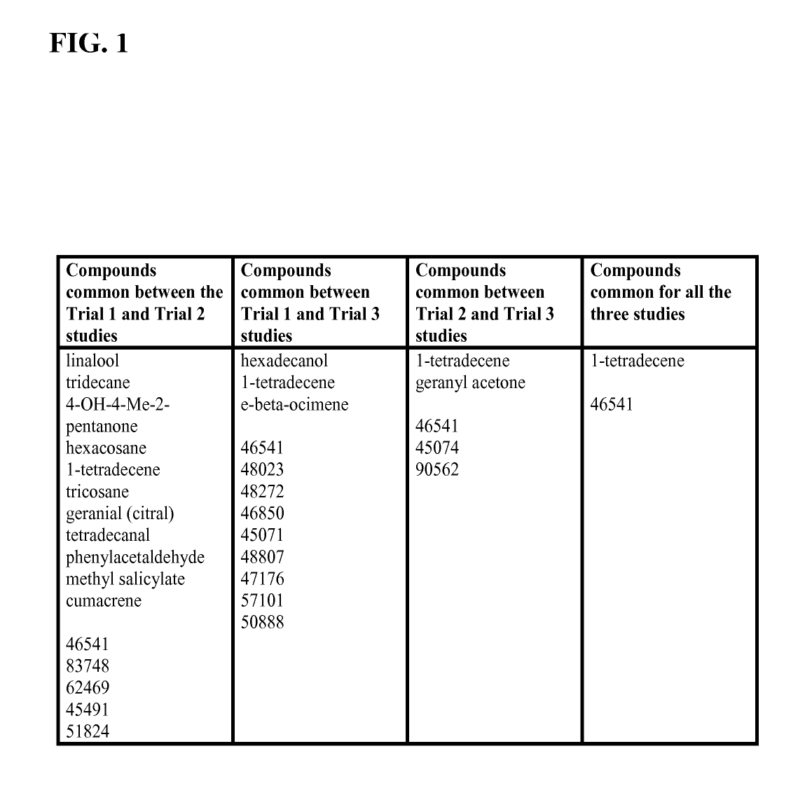

To further constrain the list of compounds from season-specific HLB biomarkers to those that can potentially discriminate uninfected and infected plants across different growing seasons, the list of biomarkers was narrowed down to only those that were found to discriminate the infection status during more than one season (more than one trial). The list of these biomarkers is given in FIG. 1 (the unidentified compounds are given as Fiehn database entries).

Screening D. citri Chemosensory Proteins with Compound Mixtures Using the Attenu Assay System

One of D. citri odorant binding proteins (DcOBP1) and four sensory appendage proteins (DcSAP1, DcSAP2, DcSAP3, and DcSAP4) were investigated. These chemosensory proteins have been identified, expressed and characterized by Inscent, Inc. Protein binding of odorants was investigated using the Attenu assay, a proprietary high-throughput assay system developed at Inscent, Inc. that builds upon proven fluorescence-based techniques. Attenu is a fluorescence-based competition assay that relies on detectable fluorescence quenching to identify interactions between insect chemosensory proteins and their ligands (Pelosi et al., 2006; Biessmann et al., 2010). When a ligand displaces a Fluor (fluorescent dye) from the binding pocket of an insect chemosensory protein, the resulting reduction in fluorescence signifies a binding event between the protein and that ligand. Thus, the assay allows high-throughput identification of whether a chemical of interest can be potentially detected at the peripheral nervous system level of an insect. The assay was used to screen the binding efficacy of the selected compounds. Typical assay conditions utilize ˜2 μM-5 μM of binding protein and 2 μM-10 μM of selected chemical compounds.

The goal of the screen was to determine which, if any, of the specific chemicals or mixtures identified could bind to either of the five chemosensory proteins from D. citri available at Inscent, Inc.: DcOBP1, DcSAP1, DcSAP2, DcSAP3, and DcSAP4. Each protein was screened at 4 μM (DcOBP1) and 10 μM (DcSAP1, DcSAP2, DcSAP3, and DcSAP4) correspondingly with every compound that was available (Table 1), as well as an equimolar mixture of compounds, wherein each compound was present at 10 μM. Further screening of the compound mixtures that mimic biogenic abundances was conducted with the total concentration of all components at 10 μM, with ratios of compounds corresponding to the values given in Table 1. The chemosensory proteins were assayed at ˜4 μM-10 μM within DMSO as the solvent. Screening for binding was performed in triplicate and with appropriate controls in order to confirm possible significant interactions. The amount of tricosane greatly exceeded that of the other components in the mixture. To assess the response to the blend of low abundance components, a second series of trials was conducted for each protein with tricosane omitted from both uninfected and CLas-infected samples. Each protein was screened with each mixture eight times. A positive control ligand (indole) was used to verify each protein was functional under assay conditions.

Behavioral Bioassays

A two-port divided T-olfactometer (Analytical Research System, Gainesville, Fla.) was used to evaluate the behavioral response of D. citri to infected (′HLW) and uninfected (‘Healthy’) odorant mixtures. Chemicals were obtained from the commercial sources as described above. The olfactometer consisted of a vertical 30 cm long glass tube with 3.5 cm internal diameter that is bifurcated into two equal halves with a Polytetrafluoroethylene (PTFE) strip forming a T-maze. Each half served as an arm of the olfactometer enabling the D. citri to make a choice between two potential odor fields. The chambers containing treatments were attached to inlet and outlet valves for incoming and outgoing air streams, respectively. Purified and humidified air was pushed through these chambers via two pumps connected to an air delivery system at 0.1 L/min flow (ARS, Gainesville, Fla.). A female D. citri was released into the olfactometer and given a choice between two odor sources for 5 minutes. D. citri were considered non-responsive if they did not make a choice within 5 min. Odor sources were randomly assigned to one arm of the olfactometer at the beginning of each bioassay and were reversed every five insects to eliminate positional bias. In addition, prior to odor testing, D. citri adult females were exposed to clean air vs. clean air in the olfactometer to verify the absence of positional bias. Response of D. citri between the two odor arms in each choice test was assessed with the use of a chi-squared test with an even distribution between odor arms as a null hypothesis.

Behavioral Response of D. citri

The objective of this experiment was to determine whether the ‘HLB’ blend was attractive to D. citri when presented against the ‘Healthy’ blend. The odor sources consisted of 24 cm length volatile collection chambers from Analytical Research Systems (Gainesville, Fla.) as described by Mann et al. (2011) enclosing a 4 cm cotton wick. Each cotton wick was impregnated with 100 μL of either ‘HLB’ or ‘Healthy’ blend at 0.1 or 0.01 μg/μL concentrations. Blends were tested at each concentration against solvent (control) and against each other at both concentrations. This test consisted of 6 to 8 trials of 15 to 21 females resulting in a total of 105 to 165 females tested per treatment combination.

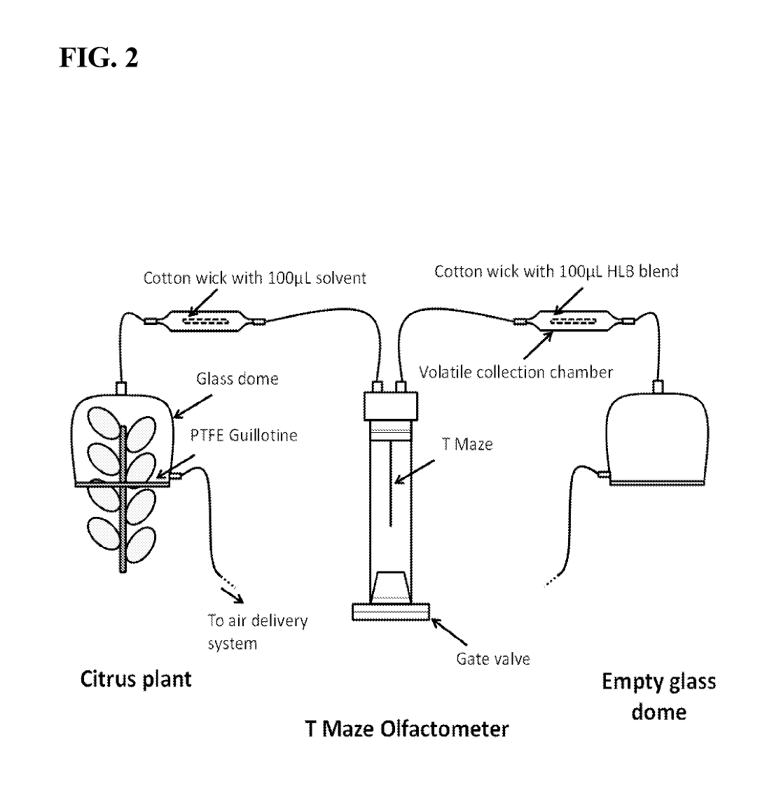

The objective of the second experiment was to determine whether the infected (‘HLB’) blend was attractive to D. citri when presented against odors from uninfected sweet-orange plants (Citrus sinensis (L.) Osbeck). Each odor arm consisted of a glass dome (38 cm tall, 15 cm width, 5 L) placed on a PTFE guillotine and attached to volatile collection chambers used in the first behavioral experiment (FIG. 2). One arm of the olfactometer received air from a clean and empty glass dome while the collection chamber contained a 4 cm cotton wick impregnated with 100 μL of the ‘HLB’ blend at the 0.1 μg/μL dosage (FIG. 2). The other arm received odors from a glass dome containing a sweet-orange Valencia plant while the collection chamber simultaneously contained a 4 cm cotton wick with 100 μL of dichloromethane (solvent blank) (FIG. 2). The plants were 2-years old, between 65 and 90 cm in height, and pruned 10 days prior to experiments to induce new leaf growth. The PTFE guillotine dome contained between 160 and 200 cm² of leaf surface and leaf flush were approximately 2.5 and 5 cm in size within the dome. The leaf surface was calculated according to a standard curve based on the length of each leaf. This test consisted of eight plants used for trials of 20 females, resulting in a total of 160 females tested.

### Results

Selection of Chemoattractant Compounds

There were 245 statistically discriminating compounds between uninfected and infected trees, based on abundance, identified in the Trial 1 samples; 82 discriminating compounds in the Trial 2 samples; and 38 discriminating compounds in the Trial 3 samples. These compounds overlapped to a large extent with the HLB biomarker compounds reported by Aksenov et al. (2014).

Partial least squares (PLS) regression analysis was applied to quantitatively examine the discrimination power of the selected compounds using a 5-fold cross-validation strategy (Wold et al. 2001). The systematic classification accuracies between uninfected and infected plants, based on these compounds, were found as follows: 95.0% (53 correctly classified out of 57 for CLas-infected and 62 correctly classified out of 64 for uninfected) for the Trial 1 samples; 83.5% (12 correctly classified out of 20 for uninfected and 54 correctly classified out of 59 for CLas-infected) for the Trial 2 samples; 83.3% (8 correctly classified out of 12 for uninfected and 22 correctly classified out of 24 for HLB-infected) for the Trial 3 samples. Thus, these discriminating compounds may be considered as ‘general purpose’ HLB biomarkers, since differences in their abundances were indicative of infection with the CLas pathogen and enabled determining the infection status of citrus trees for all infection stages with relatively high accuracy.

Approximately half of the compounds in FIG. 1 were identified and selected for developing a potential blend attractive to D. citri. The list of these compounds, along with their averaged experimental abundances, is given in Table 1. For discrimination of uninfected and infected trees, based only on the abundances of these common compounds given in FIG. 1, the classification accuracy remains nearly unchanged. For example, 93.4% (54/57 for HLB-infected and 59/64 for uninfected) for the Trial 1 samples and 83.5% (10/20 for uninfected and 56/59 for CLas-infected) for the Trial 2 samples. These results indicate that this ‘universal’ biomarker subset, although very limited compared to the total number of volatiles produced by trees, is still sufficient to discern CLas infection with high accuracy.

TABLE 1
Common subset of chemical compounds differentially
expressed in uninfected and infected citrus
plants during different seasons (trials)

| | Experimental abundances, | | | |
|---|---|---|---|---|
| | au¹ × 10⁻⁴ | | Ratios in the mix² | |
| | Uninfected | Infected | Uninfected | Infected |
| Linalool | 8.75 | 12.5 | 11.16 | 11.81 |
| Tridecane | 5.28 | 6.63 | 6.73 | 6.28 |
| 4-OH-4-Me-2- | 1.21 | 1.06 | 1.54 | 1.00 |
| Pentanone | | | | |
| Hexacosane | 8.34 | 6.53 | 10.64 | 6.18 |
| 1-Tetradecene | 4.03 | 6.34 | 5.14 | 6.01 |
| Tricosane | 9.24 | 64.3 | 117.83 | 60.87 |
| Geranial (Citral) | 1.79 | 10.0 | 2.28 | 9.48 |
| Tetradecanal | 2.52 | 7.61 | 3.22 | 7.21 |
| Phenylacetaldehyde | 6.45 | 8.95 | 8.23 | 8.48 |
| Methyl Salicylate | 5.11 | 13.2 | 6.53 | 12.46 |
| Cumacrene³ | 0.78 | 3.69 | 1.00 | 3.50 |
| (E)-Beta-Ocimene | 8.37 | 5.36 | 10.68 | 5.08 |
| Hexadecanol | 1.21 | 1.06 | 1.54 | 1.00 |
| Geranyl Acetone | 25.8 | 46.1 | 32.95 | 43.67 |

¹Values are denoted as arbitrary units based on mass spectrometry readout.
²Values are relative to the lowest abundance compound identified.
³Pure compound was not available.

Screening D. citri Chemosensory Proteins with Compound Mixtures Using the Attenu Assay System

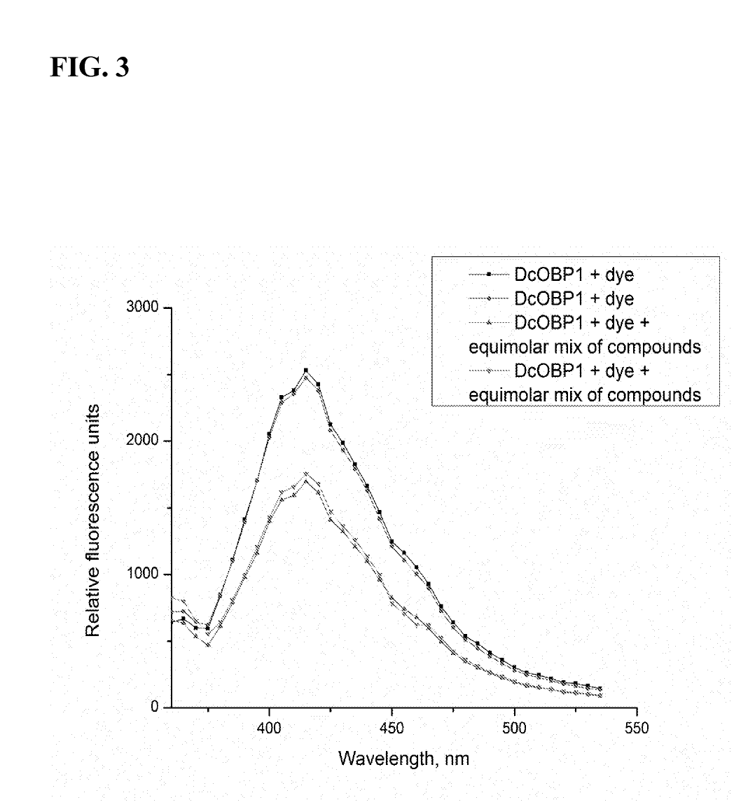

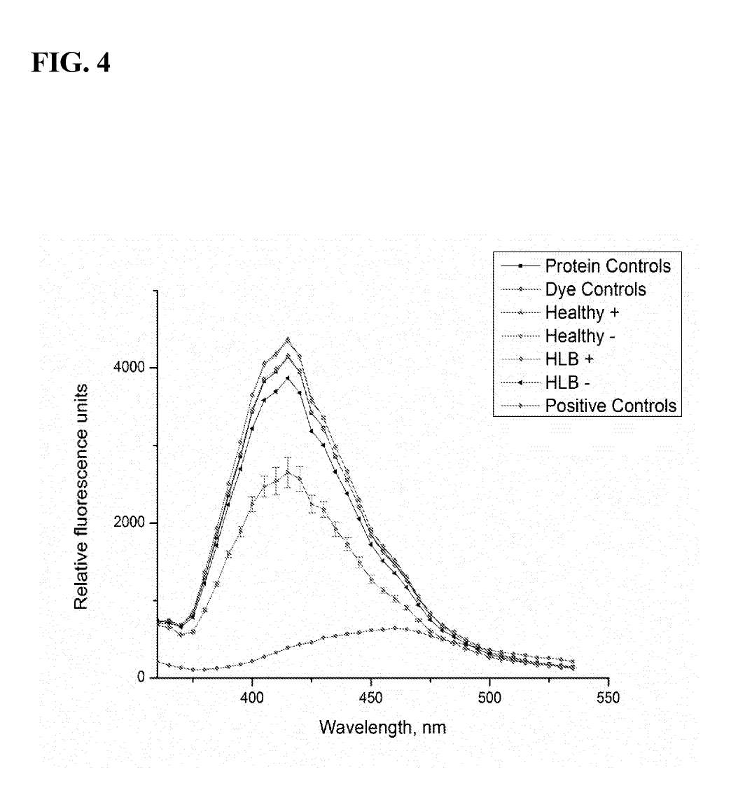

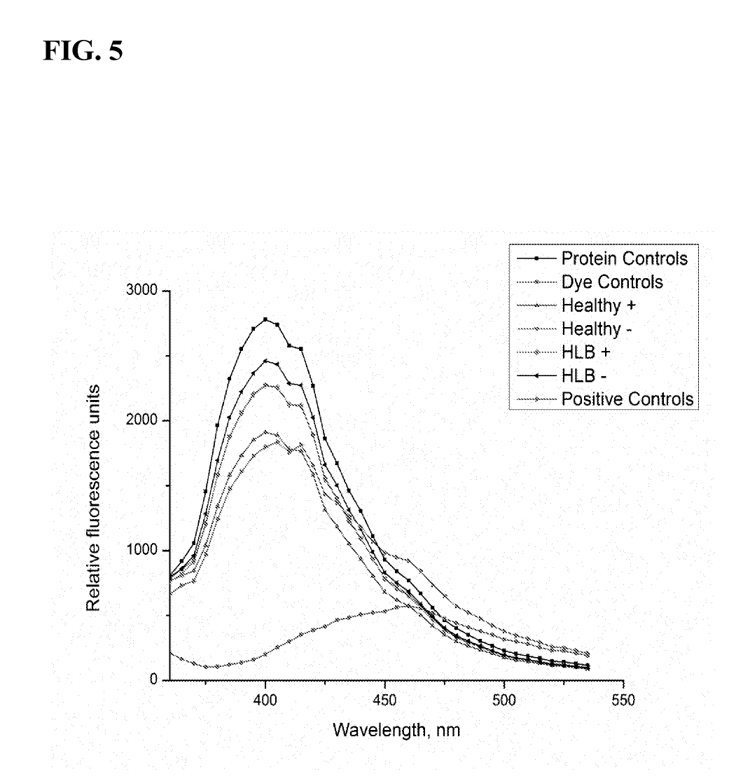

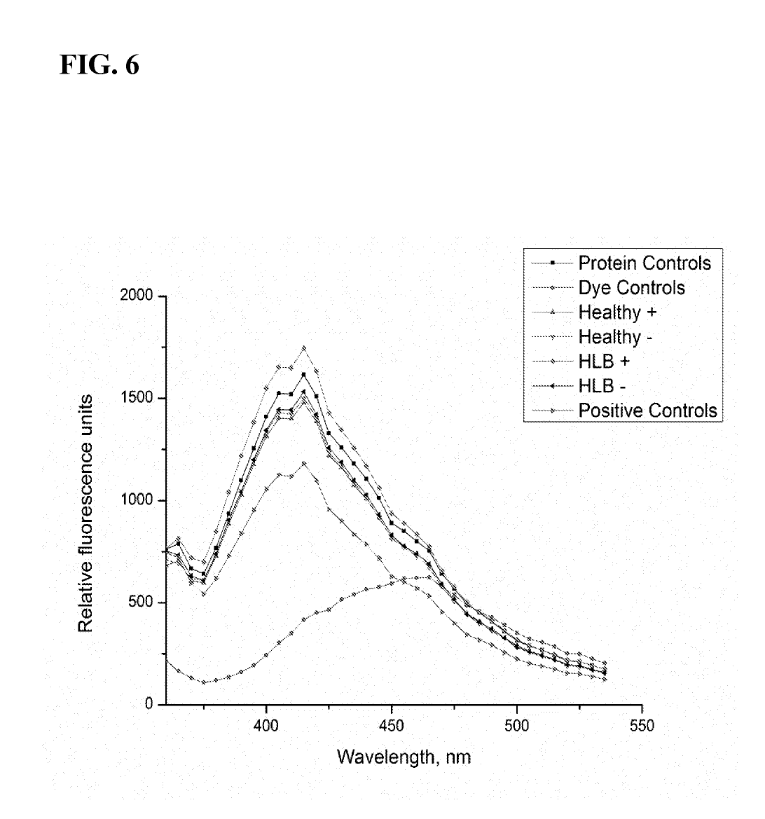

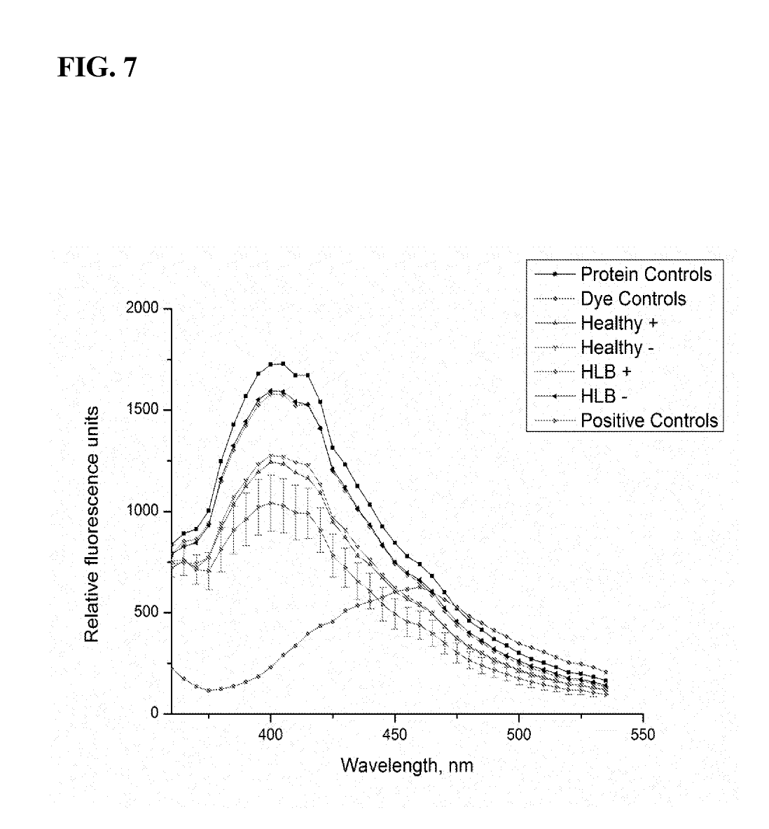

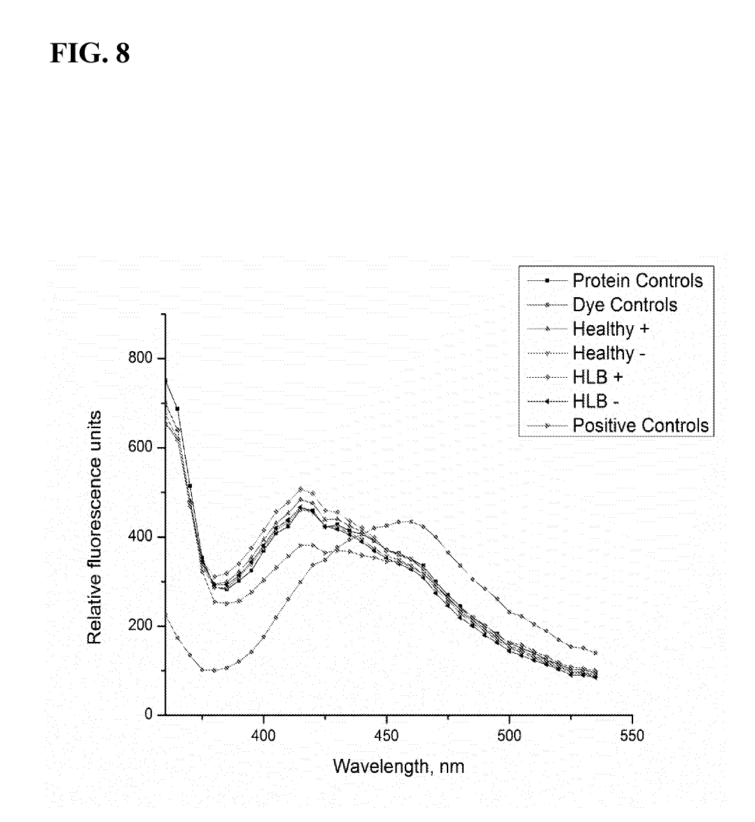

The Attenu assays indicated that none of the molecules bound any of the D. citri chemosensory proteins, when tested individually. However, the equimolar mixture of all compounds did show moderate binding to DcOBP1 (FIG. 3). Mixtures were created with ratios of compounds as listed in Table 1 (total concentration of all compounds 10 μM) with the intention to represent typical semiochemical emissions from uninfected or infected trees under assay conditions. The assay results are shown in FIG. 4, FIG. 5, FIG. 6, FIG. 7, and FIG. 8. The results did not indicate interactions between the mixtures and DcOBP1 (FIG. 4). DcSAP1 responded strongest to the uninfected (‘Healthy’) plant mixture with tricosane, and to a lesser extent to the ‘infected’ mixture with tricosane (FIG. 5). DcSAP2 did not respond strongly to any mixture, but showed potential weak interactions with the uninfected (‘Healthy’) mixture, both in the presence and absence of tricosane (FIG. 6). DcSAP3 bound the uninfected (‘Healthy’) mixture both in the presence and absence of tricosane (FIG. 7). DcSAP4 did not interact with any of the mixtures tested (FIG. 8).

Behavioral Bioassays

The behavioral response of D. citri to these artificial chemical blends was tested with a vertical T-maze olfactometer (Mann et al., 2011). Single D. citri females were introduced into the olfactometer and were given the choice between two odor arms. Female D. citri were not attracted to the uninfected (‘Healthy’) blend as compared to the solvent blank at both dosages tested (0.01 μg/μL: χ=1.15, n=105, d.f.=1, p=0.283; 0.1 μg/μL: χ=0.82, n=78, d.f.=1, p=0.365). Female D. citri were not attracted to the infected (‘HLB’) blend at 0.01 μg/μL (χ=0.07, n=129, d.f.=1, p=0.792), but were attracted to this blend at the 0.1 μg/μL dosage (χ=5.04, n=134, d.f.=1, p=0.025) as compared with the solvent blank.

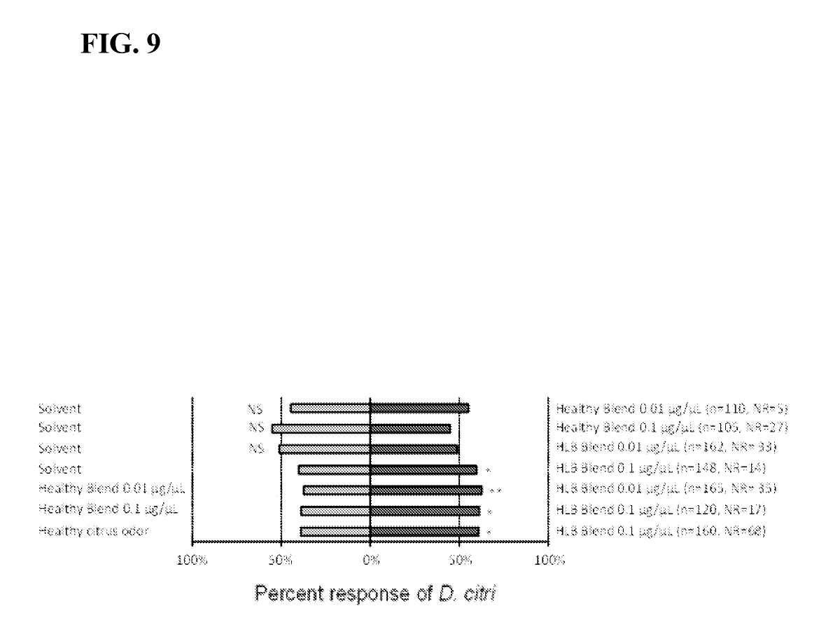

Female D. citri were consistently attracted to the infected (‘HLB’) blend as compared with the uninfected (‘Healthy’) blend at both the 0.01 μg/μL (χ=8.31, n=131, d.f.=1, p=0.004) and the 0.1 μg/μL (χ=5.14, n=103, d.f.=1, p=0.023) dosages. Similarly, D. citri were significantly more attracted to the synthetic infected (‘HLB’) blend than the natural odor from uninfected sweet orange citrus trees (χ=4.35, n=92, d.f.=1, p=0.037). Only the synthetic ‘FMB’ blend attracted D. citri as compared with a blank solvent at the concentrations tested here. Also, D. citri were consistently attracted to the synthetic, multi-component ‘HLB’ blend as compared with odors consistent with uninfected plants. This occurred when response of D. citri was compared between the ‘FMB’ synthetic blend versus a synthetic blend mimicking uninfected trees (FIG. 9). Also, D. citri were more attracted to the synthetic ‘FMB’ blend than to odors from uninfected citrus trees serving as the controls (FIG. 9).

### Discussion

Applicants attempted to develop an attractant for D. citri by identifying and mimicking the chemical cues produced by HLB-infected citrus trees. The chemical signature of sweet orange citrus trees infected with the CLas pathogen that causes HLB has been previously elucidated (Aksenov et al., 2014). The VOC profiling of citrus plants was conducted throughout an entire year in three independent studies across different growing seasons (Trials 1-3). It was demonstrated that overall VOC distributions changed significantly by season (each trial was in a different season). Both uninfected and CLas-infected trees were characterized by complex VOC ‘landscapes’ (Aksenov et al., 2014). The compounds with different abundances among uninfected and HLB-infected plants (potential HLB biomarkers) were also found to be season-specific in release. Seasonal changes in plant metabolism coupled with fluctuating titer of CLas (Manjunath et al., 2008) and differences in the pathogen's life cycle [e.g. differential expression of CLas genes (Yan et al., 2013)] may contribute to annual changes in VOC profiles. It would be challenging and likely impractical to create such complex and dynamic mixtures of hundreds of compounds for use as insect lures. However, without wishing to be bound by theory, it was thought that only a subset of compounds emitted by citrus plants would be necessary to affect D. citri behavior.

Applicants focused on a short list of compounds that were selected as ‘universal’ HLB biomarkers (Table 1, FIG. 1) for assaying behavior of D. citri. Developing a lure blend with only a subset of compounds is practical, but it is a limited representation of the entire citrus VOC profile, especially since almost a half of compounds in FIG. 1 were not identified. However, the identified compounds in this subset were previously implicated as D. citri attractants, most notably, methyl salicylate (MeSA) (Mann et al., 2012). Some of the identified compounds in Table 1 (e.g. linalool, β-ocimene) were identified as semiochemicals attractive to D. citri as they are produced by young leaf flush of rutaceous plants. Since young leaves are the primary feeding and egg laying resource for D. citri (Patt and Setamou, 2010), without wishing to be bound by theory, it is presumed that D. citri preferentially select young leaves using olfactory and visual cues. Thus, alteration of abundances of these compounds due to Clas infection may be responsible for even greater attraction of D. citri to critical resources on infected plants (Eigenbrode et al., 2002; McLeod et al., 2005; Mauck et al., 2010b; Davis et al., 2012; Shapiro et al., 2012).

In order to investigate whether the selected compounds may be active at the peripheral level, each of the 14 compounds given in Table 1 was tested with the Attenu assay system (Pelosi et al., 2006) with five chemosensory proteins from D. citri, as described above. The assay revealed that no individual compound interacted with any of the tested proteins. One interpretation is that the available chemosensory proteins were not capable of binding these particular individual compounds in vivo or the response was not elicited by a single compound and, perhaps, binding of multiple compounds was required. To explore the latter possibility, an equimolar mixture of all 14 compounds was tested to all five available odorant binding proteins. In the case of DcOBP1, a moderate interaction between the proteins and the compound mixture was observed (FIG. 3). Consequently, the mixtures intended to represent typical semiochemical emissions from uninfected and infected trees were also tested. It was found that the mixtures of biomarkers with concentrations approximately corresponding to their gas-phase abundances in the field studies produced a response from more than one chemosensory protein (FIG. 4-FIG. 8). The assay screening results indicated that a mixture of semiochemicals allowed for the selected chemical biomarkers to elicit an odorant binding response in D. citri. Typically numerous ligands, both natural and/or synthetic, may produce response for any given OBP. There are several examples of crystal structures of OBP proteins, and dimerization of OBPs has also been noted (Briand et al., 2001; Wogulis et al., 2006; Leite et al., 2009; Tsitsanou et al., 2013). Without wising to be bound by theory, these dimers may affect individual component conformations, as well as lead to the formation of a “third” binding pocket. The above factors may have contributed to the observed effect.

The Attenu technique allowed for rapid screening of functionality of a complex blend of semiochemicals based on volatile changes that occurred because of pathogen infection. The elicited response appears to be more pronounced for the uninfected (‘Healthy’) blend as compared to the infected blend. However, the results of this assay could only be interpreted as to whether the insect's odorant binding proteins interacted with the semiochemical blend. Preferential attraction of D. citri to the infected blend as compared with the uninfected blend indicates the importance of particular abundance ratios of semiochemicals for attraction of D. citri to pathogen-infected plants.

MeSA alone is an attractant for D. citri (Mann et al 2012). However, under field conditions, D. citri must discriminate among bouquets of volatiles and complex mixtures are likely more important for host location than individual compounds (Webster et al. 2010). Therefore, a mixture of compounds that identifies HLB-infected plants is likely important for location of infected hosts by D. citri. The current research indicates that uninfected trees released less MeSA as compared with infected counterparts. This suggests that MeSA induction is not restricted to HLB infection and likely not the sole indicator of infected plants for D. citri. For example, herbivore damage is known to cause release of MeSA in citrus (Mann et al. 2012, Martini et al. 2014) and other causes of damage or stress may also induce release of this volatile. Consequently, it is suggested that a complex blend of chemicals, rather than MeSA alone, may be more important to D. citri for selecting an infected plant in the field where psyllids are challenged by complex mixtures of volatiles simultaneously.

The blends identified from both uninfected and infected plants were composed of the same compounds with only relatively small differences in abundances of constituents (˜1.5 to 2-fold, ˜4-fold for geranial). Yet, these small differences caused specific attraction of D. citri to the blend characteristic of infected plants. The idea of implementing semiochemicals for insect management is common, with significant efforts focused on insect monitoring and pheromone-based mating disruption (Witzgall et al., 2010a). Indirect control of pest populations through the use of herbivore-induced plant volatiles to attract carnivorous arthropods has also shown promise (Kaplan, 2012). The idea of “attract and reward” for conservation biological control has also been investigated (Gordon et al., 2013). Applicants present an approach for semiochemical identification that considers analytical chemistry, protein binding assays at the peripheral nervous system level, and behavioral assays. This approach has been shown feasible for citrus and D. citri, and a similar approach may be useful for developing lures for vectors of other phytopathogens. The currently identified attractive blend has use for monitoring or attract-and-kill of D. citri.

### Example 2: Statistical Analysis of Attenu Assays

This Example elaborates on the data presented in Example 1 and provides the average values (±SE) of the areas under the fluorescence curves for the Attenu assays as described in Example 1 (See FIG. 4, FIG. 5, FIG. 6, FIG. 7, and FIG. 8).

Screening D. citri Chemosensory Proteins with Compound Mixtures Using the Attenu Assay System

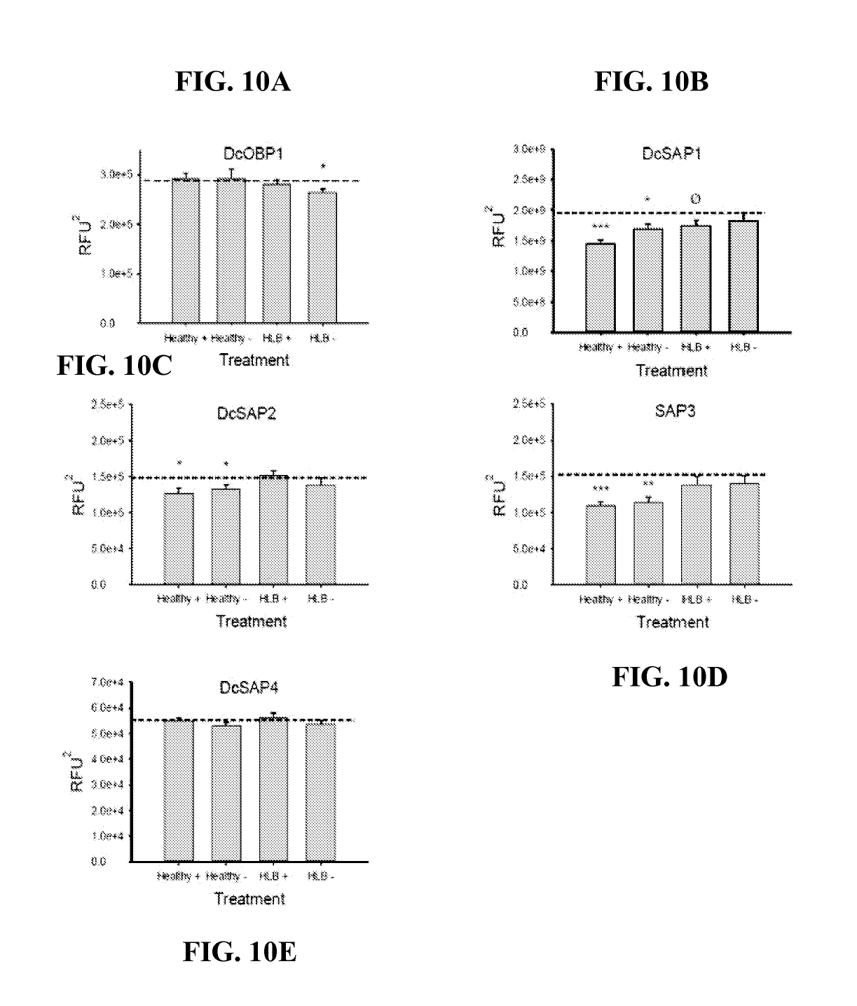

The Attenu assays indicated that none of the compounds bound to any of the D. citri chemosensory proteins when tested individually (data not shown). However, from Example 1, the equimolar mixture of all compounds did show moderate binding to DcOBP1 (FIG. 3). Mixtures were created with ratios of compounds as listed in Table 1 (total concentration of all compounds 10 μM) with the intention to represent typical semiochemical emissions from uninfected or infected trees under assay conditions. The summary of assay results is shown in FIG. 4-FIG. 8 and elaborated upon in Example 1. The results indicate interaction between the “infected” mixture without tricosane and DcOBP1 [t₍₇₎=3.37, P=0.012; FIG. 10A, FIG. 4). DcSAP1 bound strongest the uninfected (“Healthy”) plant mixture both in the presence [t₍₇₎=8.38, P<0.001] and absence of tricosane [t₍₇₎=3.074, P=0.018], and to a lesser extent the “infected” mixture with tricosane [t₍₇₎=2.31, P=0.054; FIG. 10B, FIG. 4 and FIG. 5). DcSAP2 showed interaction with the uninfected (“Healthy”) mixture, both in the presence [t₍₇₎=2.80, P=0.027] and absence of tricosane [t₍₇₎=2.62, P=0.034; FIG. 10C, FIG. 6]. DcSAP3 bound the uninfected (“Healthy”) mixture both in the presence [t₍₇₎=8.92, P<0.001] and absence of tricosane [t₍₇₎=5.15, P=0.001; FIG. 10D, FIG. 7]. DcSAP4 did not interact with any of the mixtures tested (FIG. 10E, FIG. 8).

### Example 3: SB 0.6 Formulation for Synthetic Lures of Asian Citrus Psyllids

This Example describes an additional formulation for a synthetic chemical lure which may be used as an attractant of Asian citrus psyllids (ACP). This formulation reconstitutes the odor of HLB-infected trees and is attractive to ACP in laboratory conditions. This formulation is also more attractive to ACP than the odor of a non-HLB infected citrus tree.

### Materials and Methods

The Materials and Methods used in this Example, unless stated otherwise, may be found in Example 1 above.

### Results

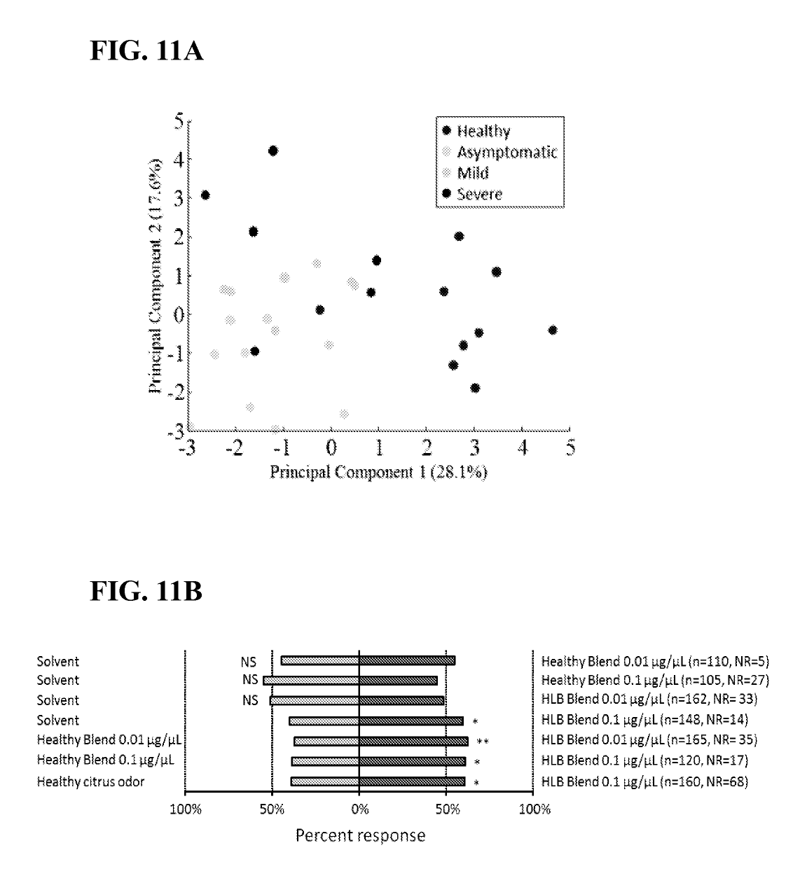

From Example 1, Applicants described a synthetic chemical lure that was an attractant of ACP (See FIG. 9, FIG. 11A, FIG. 11B). As can be seen in FIG. 11A, odors from HLB-infected trees and odors from healthy trees differed significantly. The artificial synthetic blend was also found to outperform (e.g. to be more attractive to ACP) than the odor of a healthy tree.

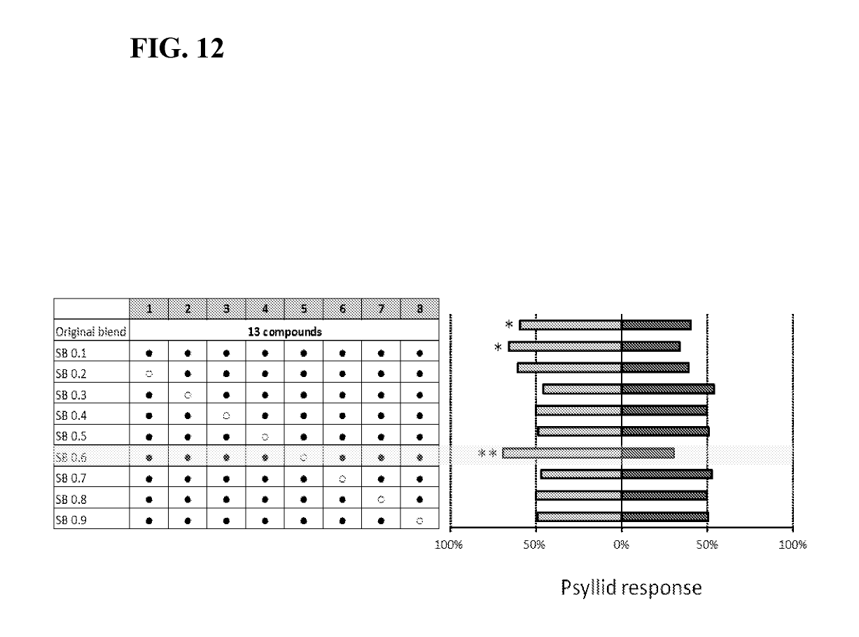

In efforts to improve the synthetic blend above, compound subtraction experiments were performed to identify core components that are the strongest attractants of ACP. The identity of the individual compounds in the new formulations tested are presented in FIG. 12, left-hand panel. The behavioral response of ACP (using a similar assay as described in FIG. 9) to each of these new formulations is presented in FIG. 12, right-hand panel. From these subtraction assays to develop improved formulations, a formulation referred to as “SB 0.6” was identified as the strongest attractant of ACP. SB 0.6 contained the following compounds: (E)-Beta-Ocimene, Tricosane, Phenylacetaldehyde, Linalool, Geranyl Acetone, Geranial, and Methyl Salicylate.

### Example 4: Additional Formulations for Synthetic Lures of Asian Citrus Psyllids

This Example describes additional formulation for synthetic chemical lures which may be used as attractants of Asian citrus psyllids (ACP). These formulations contain 7 active chemical compounds for the luring of ACP, and three of these compounds have been identified as having a major biological activity. These new blends attracted up to 70% of ACP, with a response rate over 95%.

### Materials and Methods

The Materials and Methods used in this Example, unless stated otherwise, may be found in Example 1 above.

### Results

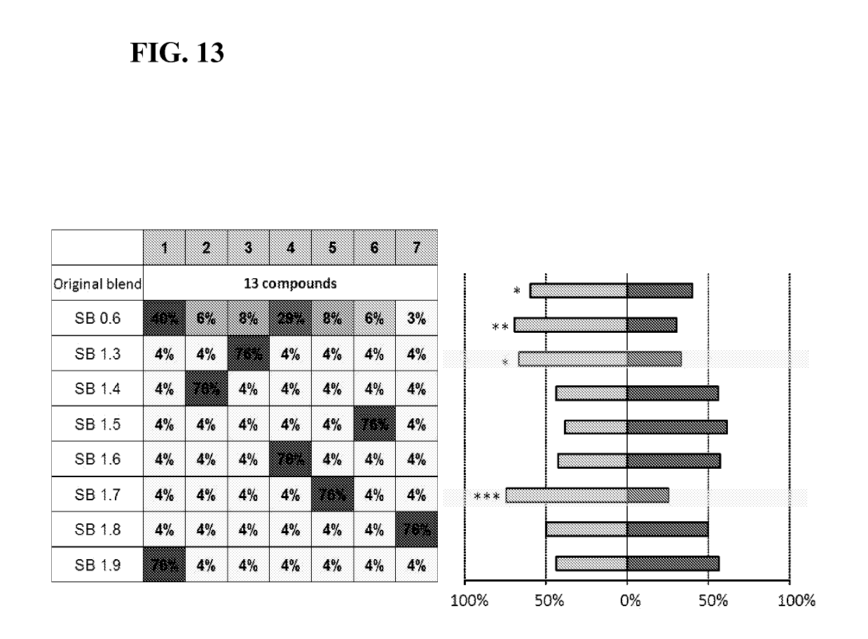

In efforts to develop improved synthetic chemical lures of ACP, different formulations containing different percentages of seven compounds were tested for their ability to attract ACP. In these experiments, formulations were tested that contained a single “major” active component, and a series of additional “minor” active components (SB 1.3-SB 1.9). The results of these assays are presented in FIG. 13. It was found that blends where Methyl salicylate and Linalool were the “major” compounds gave the best results (e.g. were the most attractive to ACP).

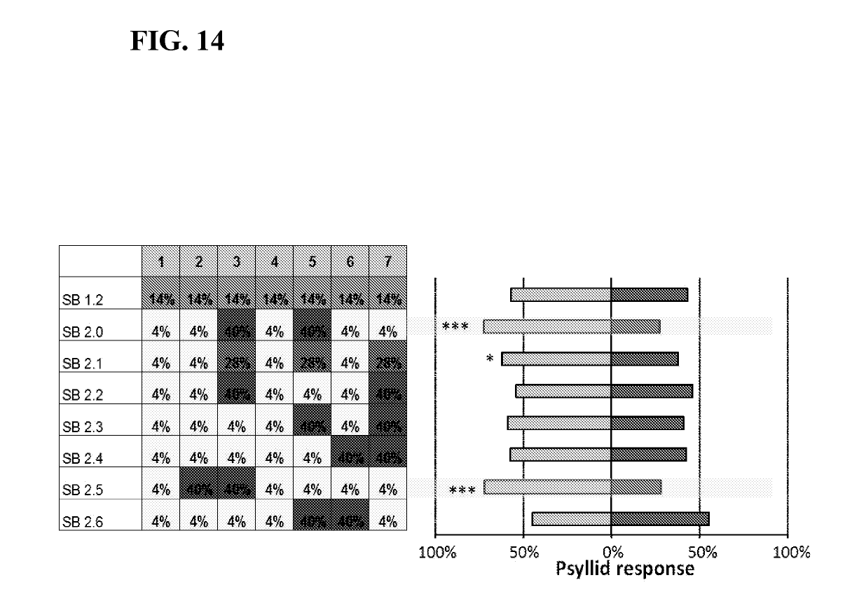

Additional chemical lures of ACP were also tested, these lures having different formulations containing different percentages of seven compounds and being tested for their ability to attract ACP. The formulations tested in these experiments were named SB 1.2-SB 2.6. The results of these assays are presented in FIG. 14. It was found that blends where Methyl salicylate, Linalool, and Geranial were dominant gave the best results (e.g. were the most attractive to ACP). Notably, for blend 2.0 (SB 2.0), the percentage of psyllid attracted by the blend was 70%, the percentage of psyllid that responded was 96%, and the time for response was 42 seconds.

### Example 5: Field Trial 1-Test of ACP Attraction to Artificial Blends

This Example describes the results of a field trial testing the ability of various synthetic chemical lures to attract Asian citrus psyllids (ACP) in field conditions.

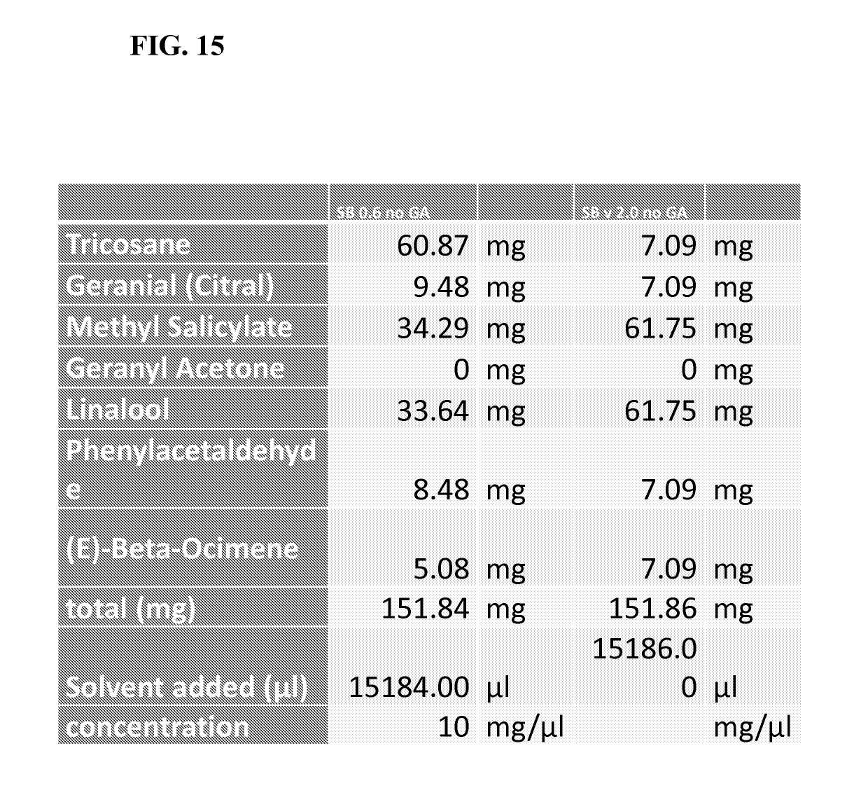

The purpose of this study was to determine the optimal dose of attractive volatiles for ACP. Preliminary work with attractant volatiles (isolated from HLB infected host trees) elucidated a seven component blend that can be used for attraction of the Asian citrus psyllid, Diaphorina citri. Two different seven-component blends, containing the same components but in different quantities, were selected for evaluation in field trials. The compositions of these two blends are presented in FIG. 15.

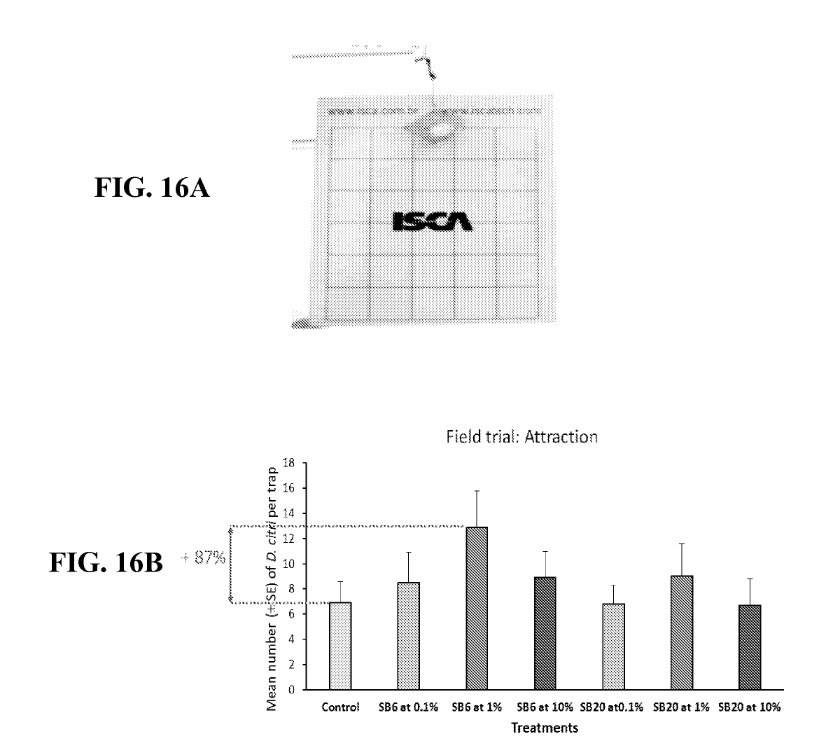

Trials were conducted in an open environment containing citrus trees. Yellow sticky traps from ISCA Technologies were used as the trap device to capture ACP in these trials, as shown in FIG. 16A. The base technology used to test these formulations in field trials is known as SPLAT™ (Specialized Pheromone & Lure Application Technology)(www.iscatech.com/exec/SPLAT.html).

The results of the field trials with the two chemical blends are presented in FIG. 16B. Overall, it was found that SB 0.6 was the best blend tested in the field. SB 0.6 at 1% was able to produce an 87% increase in the average number of psyllids present on the trap over the control.

### Example 6: Field Trial 2-Test of ACP Attraction to Artificial Blends Containing an Insecticide

This Example describes the results of a field trial testing the ability of various synthetic chemical lures that also contain an insecticide to attract Asian citrus psyllids (ACP) in field conditions.

The purpose of this study was to determine whether deployment of an attractive blend of volatiles can be used to effectively attract ACP adults to dollops, and how this can be used to enhance population control and crop protection. The efficiency of SPLAT™ application with two attractants was tested, one from the paper from Coutinho-Abreu et al. (2014) and the other with the blend SB 0.6, based on previous field and lab trials. The exact composition of the formulations tested in the field trial are presented below.

Experimental matrix formulation containing 1.66% Myrcene+1.66% Ethyl butyrate+1.66% p-cymene (˜5% total attractant AI)+0.2% Spinosad. Formulation 1: XF2001K01

Experimental matrix formulation containing 1% total attractant AI (40% Tricosane+22.6% Methyl Salicylate+22.2% Linalool+6.2% Geranial+5.6% Phenylacetaldehyde+3.4% E(B)-ocimene+0.2% Spinosad). Formulation 2: XF2001K02

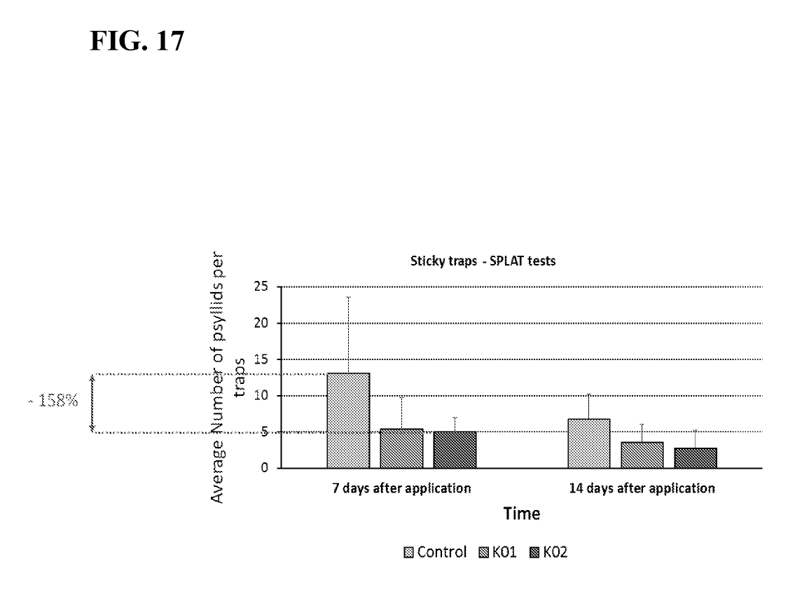

The dosage of the formulations above that were tested in the field trial was as follows: 8 dollop (2.5 g) per tree. The results of this field trial are presented in FIG. 17. It should be noted that tests at 4 dollop per tree were not effective (data not shown).

### REFERENCES

Aksenov et al. (2014). Detection of Huanglongbing disease using differential mobility spectrometry. Anal. Chem. (Washington, D.C., U. S.) 86, 2481-2488. doi: 10.1021/ac403469y.
Biessmann et al. (2010). The Anopheles gambiae Odorant binding protein 1 (AgamOBP1) mediates indole recognition in the antennae of female mosquitoes. Plos One 5. doi: 10.1371/journal.pone.0009471
Briand et al. (2001). Ligand binding and physico-chemical properties of ASP2, a recombinant odorant-binding protein from honeybee (Apis mellifera L.). Eur. J. Biochem., 268, 752-760.
Callaway. (2008). Bioterror: The green menace. Nature 452, 148-150. doi: 10.1038/452148a.
Davis et al. (2012). Experimental infection of plants with an herbivore-associated bacterial endosymbiont influences herbivore host selection behavior. PLoS ONE 7:e49330. doi: 10.1371/journal.pone.0049330
Eigenbrode et al. (2002). Volatiles from potato plants infected with potato leafroll virus attract and arrest the virus vector, Myzus persicae (Homoptera: Aphididae). Proc. R. Soc. B. 269, 455-460. doi: 10.1098/rspb.2001.1909
El-Sayed. (2012). The Pherobase: Database of Pheromones and Semiochemicals. Available at the “www.pherobase.com” website.
Gordon et al. (2013). ‘Attract and reward’: Combining a herbivore-induced plant volatile with floral resource supplementation—Multi-trophic level effects. Biol.1 Control 64, 106-115. doi: 10.1016/j.biocontrol.2012.10.003.
Grafton-Cardwell et al. (2013). Biology and management of Asian citrus psyllid, vector of the Huanglongbing pathogens. Annu. Rev. Entomol., Vol 58 58, 413-432. doi: 10.1146/annurev-ento-120811-153542.
Fiehn database, available at the “fiehnlab.ucdavis.edu/db/” website.
Inscent, available at the “www.inscent.com/”.
KaplanI. (2012). Attracting carnivorous arthropods with plant volatiles: The future of biocontrol or playing with fire? Biol. Control 60, 77-89. doi: 10.1016/j.biocontrol.2011.10.017.
Kos et al. (2013). Genetic engineering of plant volatile terpenoids: effects on a herbivore, a predator and a parasitoid. Pest Manag. Sci. 69, 302-311. doi: 10.1002/Ps.3391.
Kost and Heil. (2006). Herbivore-induced plant volatiles induce an indirect defense in neighbouring plants. J. Ecol. 94, 619-628. doi: 10.1111/j.1365-2745.2006.01120.x.
Leite et al. (2009). Structure of an odorant-binding protein from the mosquito Aedes aegypti suggests a binding pocket covered by a pH-sensitive “Lid”. PLoS One, 4, e8006.
Manjunath et al. (2008). Detection of Candidatus Liberibacter asiaticus' in Diaphorina citri and its importance in the management of Citrus huanglongbing in Florida. Phytopathology, 98, 387-396. doi: :10.1094/PHYTO-98-4-0387
Mann et al. (2012). Induced release of a plant-defense volatile ‘deceptively’ attracts insect vectors to plants infected with a bacterial pathogen. PLoS Pathog. 8, e1002610. doi: 10.1371/journal.ppat.1002610.
Mann et al. (2011). Sulfur volatiles from Allium spp. affect Asian citrus psyllid, Diaphorina citri Kuwayama (Hemiptera: Psyllidae), response to citrus volatiles. B. Entomol. Res. 101, 89-97. doi: 10.1017/S0007485310000222.
Martini et al. (2014) Plant volatiles and density-dependent conspecific female odors are used by Asian citrus psyllid to evaluate host suitability on a spatial scale. Arthropod-Plant Ints. In press. doi: 10.1007/s11829-014-9326-z.
Mauck et al. (2010). Deceptive chemical signals induced by a plant virus attract insect vectors to inferior hosts. P. Nat. Aca. Sci. USA 107, 3600-3605. doi: 10.1073/pnas.0907191107.
McLeod et al. (2005). The pathogen causing Dutch elm disease makes host trees attract insect vectors. Proc. R. Soc. B. 272, 2499-2503. doi: 10.1098/rspb.2005.3202
Patt and Setamou. (2010). Responses of the Asian citrus psyllid to volatiles emitted by the flushing shoots of its rutaceous host plants. Env. Entomol. 39, 618-624. doi: 10.1603/En09216.
Pierik et al. (2014) Ecology of plant volatiles: taking a plant community perspective. Plant Cell Env. 37:1845-1853. doi: 10.1111/pce.12330
Pelosi et al. (2006). Soluble proteins in insect chemical communication. Cell. Mol. Life Sci. 63, 1658-1676. doi: DOI 10.1007/s00018-005-5607-0.
Pelz-Stelinski et al. (2010). Transmission parameters for ‘Candidatus Liberibacter asiaticus’ by Asian citrus psyllid (Hemiptera: Psyllidae). J. Econ. Entomol. 103, 1531-1541. doi: 10.1603/EC10123
Rodriguez-Saona et al. (2011). Field responses of predaceous arthropods to methyl salicylate: a meta-analysis and case study in cranberries. Biol. Control 59, 294-303. doi: 10.1016/j.biocontrol.2011.06.017
Rusconi et al. (2012). Mapping the Anopheles gambiae Odorant Binding Protein 1 (AgamOBP1) using modeling techniques, site directed mutagenesis, circular dichroism and ligand binding assays. BBA.-Proteins Proteom. 1824, 947-953.
Shapiro et al. (2012). Pathogen effects on vegetative and floral odours mediate vector attraction and host exposure in a complex pathosystem. Ecol. Lett. 15, 1430-1438. doi: 10.1111/ele.12001
Skogerson et al. (2011). The volatile compound BinBase mass spectral database. BMC Bioinformatics 12. doi:10.1186/1471-2105-12-321
Tsitsanou et al. (2013). Crystal and Solution Studies of the “Plus-C” Odorant-binding Protein 48 from Anopheles gambiae Control of binding specificity through three-dimensional domain swapping J. Biol. Chem. 288, 33427-33438.
Webster et al. (2010) Volatiles functioning as host cues in a blend become nonhost cues when presented alone to the black bean aphid. Anim Behav 79, 451-457.
Wold et al. (2001) PLS-regression: a basic tool of chemometrics. Chemometr. Intell. Lab. 58, 109-130.
Witzgall et al. (2010a). Sex pheromones and their impact on pest management. J. Chem. Ecol. 36, 80-100. doi: 10.1007/s10886-009-9737-y.
Witzgall et al. (2010b). Pheromone communication channels in tortricid moths: lower specificity of alcohol vs. acetate geometric isomer blends. Bull. Entomol. Res. 100, 225-230. doi: 10.1017/s0007485309990186.
Wogulis et al. (2006). The crystal structure of an odorant binding protein from Anopheles gambiae: Evidence for a common ligand release mechanism. Biochem. Bioph. Res. Co. 339, 157-164.
Yan et al. (2013). Global gene expression changes in Candidatus Liberibacter asiaticus during the transmission in distinct hosts between plant and insect. Mol. Plant Pathol. 14, 391-404. doi: 10.1111/mpp.12015.
Coutinho-Abreu, Iliano V., et al. (2014). Odorants for Surveillance and Control of the Asian Citrus Psyllid (Diaphorina citri). e109236.
Aksenov A A et al. (2014). Synthetic blends of volatile, phytopathogen-induced odorants can be used to manipulate vector behavior. Front. Ecol. Evol. 2:78. doi: 10.3389/fevo.2014.00078.

What is claimed is:

1. A composition comprising a solvent and a mixture of two or more compounds selected from the group consisting of linalool, tridecane, 4-OH-4-Me-2-pentanone, hexacosane, 1-tetradecene, tricosane, geranial, tetradecanal, phenylacetaldehyde, methyl salicylate, cumacrene, (E)-β-ocimene, hexadecanol, and geranyl acetone, wherein the composition is an attractant for psyllids.

2. The composition of claim 1, wherein the mixture comprises linalool, tricosane, geranial, phenylacetaldehyde, methyl salicylate, and (E)-β-ocimene.

3. The composition of claim 2, wherein the mixture further comprises geranyl acetone.

4. The composition of claim 2, wherein in the mixture, one of linalool, methyl salicylate, geranial and combinations thereof, is present at a percent weight of at least 25% of the compounds in the mixture.

5. The composition of claim 2, wherein in the mixture, tricosane, phenylacetaldehyde, and (E)-β-ocimene are each present at a percent weight of less than 10% of the compounds in the mixture.

6. The composition of claim 2, wherein the mixture constitutes less than 5% by weight of the composition.

7. The composition of claim 1, wherein the mixture comprises linalool, tridecane, 4-OH-4-Me-2-pentanone, hexacosane, 1-tetradecene, tricosane, geranial, tetradecanal, phenylacetaldehyde, methyl salicylate, cumacrene, (E)-β-ocimene, hexadecanol, and geranyl acetone.

8. The composition of claim 7, wherein in the mixture,
the molar ratio of linalool:4-OH-Me-2-pentanone is from 5 to 15,
the molar ratio of tridecane:4-OH-Me-2-pentanone is from 2 to 10,
the molar ratio of hexacosane:4-OH-Me-2-pentanone is from 2 to 10,
the molar ratio of 1-tetradecene:4-OH-Me-2-pentanone is from 2 to 10,
the molar ratio of tricosane:4-OH-Me-2-pentanone is from 30 to 90,
the molar ratio of geranial:4-OH-Me-2-pentanone is from 5 to 15,
the molar ratio of tetradecanal:4-OH-Me-2-pentanone is from 3 to 15,
the molar ratio of phenylacetaldehyde:4-OH-Me-2-pentanone is from 3 to 15,
the molar ratio of methyl salicylate:4-OH-Me-2-pentanone is from 5 to 20,
the molar ratio of cumacrene:4-OH-Me-2-pentanone is from 0.5 to 10,
the molar ratio of (E)-β-ocimene:4-OH-Me-2-pentanone is from 2 to 10,
the molar ratio of hexadecanol:4-OH-Me-2-pentanone is from 0.1 to 5, and
the molar ratio of geranyl acetone:4-OH-Me-2-pentanone is from 20 to 60.

9. The composition of claim 7, wherein in the mixture,
the molar ratio of linalool:4-OH-Me-2-pentanone is about 11.81:1,
the molar ratio of tridecane:4-OH-Me-2-pentanone is about 6.28:1,
the molar ratio of hexacosane:4-OH-Me-2-pentanone is about 6.18:1,
the molar ratio of 1-tetradecene:4-OH-Me-2-pentanone is about 6.01:1,
the molar ratio of tricosane:4-OH-Me-2-pentanone is about 60.87:1,
the molar ratio of geranial:4-OH-Me-2-pentanone is about 9.48:1,
the molar ratio of tetradecanal:4-OH-Me-2-pentanone is about 7.21:1,
the molar ratio of phenylacetaldehyde:4-OH-Me-2-pentanone is about 8.48:1,
the molar ratio of methyl salicylate:4-OH-Me-2-pentanone is about 12.46:1,
the molar ratio of cumacrene:4-OH-Me-2-pentanone is about 3.50:1,
the molar ratio of (E)-β-ocimene:4-OH-Me-2-pentanone is about 5.08:1,
the molar ratio of hexadecanol:4-OH-Me-2-pentanone is about 1:1, and
the molar ratio of geranyl acetone:4-OH-Me-2-pentanone is about 43.67:1.

10. The composition of claim 1, wherein the compounds in the mixture are present at a concentration in the range of 0.01 μg/μL to 0.1 μg/μL.

11. The composition of claim 1, wherein the solvent is dichloromethane.

12. The composition of claim 1, wherein the composition further comprises an insecticide.

13. The composition of any one of claims 1-12, wherein the psyllid is Diaphirona citri.

14. A kit comprising the composition of any one of claims 1-12, wherein the kit comprises an apparatus suitable for dispensing the composition.

15. The kit of claim 14, wherein the composition is dispensed as a liquid.

16. The kit of claim 14, wherein the composition is dispensed as an aerosol.

17. A method of attracting a psyllid, the method comprising:
a) providing an environment comprising a psyllid;
b) contacting the environment with the composition of any one of claims 1-12 at a source location so as to attract the psyllid to the composition.

18. The method of claim 17, wherein said composition is present at a stationary source location.

19. The method of claim 17, further comprising a step of monitoring the source location for contact with a psyllid.

20. The method of claim 19, comprising monitoring changes in psyllid contact with the source location over a time interval.

21. The method of claim 20, wherein changes in psyllid contact are changes in the number of psyllids contacting the composition.

22. The method of claim 17, further comprising a step of terminating a psyllid that contacts the composition.

23. The method of claim 17, wherein the environment is a citrus orchard.

24. The method of claim 23, wherein the citrus orchard is an orange tree orchard.

25. The method of claim 17, wherein the psyllid is Diaphirona citri.

26. A method of monitoring psyllid infestation, the method comprising:
a) placing the composition of any one of claims 1-12 in a citrus orchard at a source location,
b) monitoring contact of the composition by a psyllid, wherein the contact is indicative of psyllid infestation.

27. The method of claim 26, wherein the citrus orchard is an orange tree orchard.

28. The method of claim 26, wherein the psyllid is Diaphirona citri.
